(19) United States
(12) Patent Application Publication
(10) Pub. No.: US 2008/0021731 A1
(43) Pub. Date: Jan. 24, 2008

(54) METHODS AND SYSTEMS FOR MONITORING PATIENT SUPPORT EXITING AND INITIATING RESPONSE

(75) Inventor: Mark E. Rodgers, Jackson, MS (US)

Correspondence Address: WORKMAN NYDEGGER, 60 EAST SOUTH TEMPLE, 1000 EAGLE GATE TOWER, SALT LAKE CITY, UT, 84111, US

(73) Assignee: Valence Broadband, Inc., Ridgeland, MS (US)

(21) Appl. No.: 11/779,134

(22) Filed: Jul. 17, 2007

Related U.S. Application Data
(63) Continuation-in-part of application No. 11/561,263, filed on Nov. 17, 2006.
(60) Provisional application No. 60/748,376, filed on Dec. 9, 2005.
(60) Provisional application No. 60/799,041, filed on May 10, 2006.
(60) Provisional application No. 60/835,662, filed on Aug. 4, 2006.
(60) Provisional application No. 60/826,634, filed on Sep. 22, 2006.

(51) Int. Cl. G06Q 10/00

(52) U.S. Cl. 705/2

(57) ABSTRACT

Methods, systems and computer program products for monitoring patient support exiting and initiating a response to prevent or mitigate harm. One ore more cameras provide a video data stream of a patient resting on a bed, chair, wheelchair, gurney, recliner or other support. A computer system analyses the video data stream and determines the location and/or movements of the patient relative to a fixed reference (e.g., the support). A profile containing personalized support exiting data for the patient is used to accurately predict support exiting. Intervention to prevent or mitigate harm in the event support exiting is detected may include an alarm, audio/visual communication and/or direct physical intervention. Patient profiles can be updated in response to observed behavior to better predict support exiting.

### CROSS-REFERENCE TO RELATED APPLICATION

This application is a continuation-in-part of co-pending U.S. application Ser. No. 11/561,263, filed Nov. 17, 2006. That application claims the benefit of co-pending U.S. Provisional Application No. 60/748,376, filed Dec. 9, 2005, co-pending U.S. Provisional Application No. 60/799,041, filed May 10, 2006, co-pending U.S. Provisional Application No. 60/835,662, filed Aug. 4, 2006, and co-pending U.S. Provisional Application No. 60/826,634, filed Sep. 22, 2006. The disclosures of the foregoing applications are incorporated herein in their entirety.

### BACKGROUND OF THE INVENTION

1. Field of the Invention

The invention is in the field of automated methods and systems for monitoring patients in a healthcare facility in order to prevent or reduce the incidence of falling.

2. Relevant Technology

Healthcare facilities provide clinical and/or wellness health care for patients and/or residents (hereinafter collectively referred to as “patients”) residing at such facilities. Hospitals and medical clinics provide clinical health care. Assisted living and nursing homes focus primarily on wellness health care. Although patient health, safety and general well being are or should be paramount concerns for all medical and assisted living facilities, the current standard of care for these facilities does not always ensure adequate safety and care of the patient or resident.

Most facilities provide at least some monitoring and supervision of patients to ensure they are receiving proper nutrition and medicines, are kept clean, and protected from physical injury. Many facilities include a central station (e.g., a nurse station) that functions as a primary gathering and dispatch location for caregivers. From time to time, at specified intervals, or in response to a patient or resident request, a caregiver can move from the central station to a patient's location (e.g., room) and monitor or provide appropriate care.

One area of critical concern is preventing or reducing the incidence of patient falls, which can occur in a variety of circumstance but which commonly result from unauthorized or unassisted bed exiting, wheelchair exiting, and wheelchair to bed transfer. Falls often occur due to the inability of health care facilities to provide continuous, direct supervision of patients. In many cases it may not be feasible to provide round the clock supervision of every patient due to financial and/or logistical restraints. Nevertheless, without continuous direct supervision and/or a reliable system of early notification, there is simply no way for a health care provider to know when a particular patient may be engaging in behavior which places them at a high risk for a fall. Only through direct supervision and/or early notification can a caregiver even have a chance to intervene and prevent or mitigate a potentially dangerous patient action.

Notwithstanding the need to provide continuous supervision to prevent patient falls and injury, the United States, Europe, Japan and other parts of the world are currently experiencing a serious shortage of nurses, nursing assistants, doctors, and other caregivers. The shortage of caregivers will only worsen with continued aging of the U.S., European, Japanese and other populations. As the patient to caregiver ratio of a facility increases, the incidence of patient falls is also likely to increase as more patients are left unattended.

Due to continued and worsening overcrowding of orthopedic, oncology, Alzheimer's, nursing homes, and other extended care facilities, there is an acute need for new methods and systems that can better safeguard patients while also reducing facility liability, enhancing caregiver productivity, and lowering operational expenses. Although automated patient monitoring systems have been proposed, they lack feasibility and have not been implemented on a wide scale.

One example of an automated patient monitoring system is fixing an electric eye or camera on a location near where a patient is lying. An alarm might sound if a line or plane is broken by the patient. Another example involves devices that detect patient motion. Yet another proposes comparing successive images of a patient to determine patient acceleration and relative location. One particularly creative patient monitoring system claims to be able to monitor and interpret a wide variety of patient movements, including patient falls, by taking and analyzing 3-dimensional images of a patient. Of course, once the patient has already fallen, intervention to prevent the fall is impossible.

A problem with many proposed systems is they only crudely predict or determine actual patient bed exiting or other potentially dangerous movements. The result is a high level of false positives and false negatives. A high recurrence rate of false positives can become like the boy crying wolf and might be ignored by overworked caregivers. False negatives provide no early warning of patient falls.

A common problem that leads to high levels of false positives and false negatives is a “one size fits all” approach to detecting patient movements. Although people often have uniquely personal ways of getting out of bed, no attempt is made in conventional monitoring systems to understand the idiosyncratic movements and habits of a particular patient. For example, one patient might typically grasp the left handrail when commencing to bed exit while another might slide towards the foot of the bed. Persons who are left handed might exit their beds oppositely from right handed persons. Certain medical conditions might determine or alter bed exiting behavior (e.g., a person with an incision might protect against harm or pain by avoiding movements that would apply stress to the incision, even if such movements were previously used to bed exit when the patient was healthy).

In view of the foregoing, it would be an advancement in the art to provide methods and systems for monitoring patient behavior that can more accurately detect the individual behaviors and movements that are predictive of bed exiting, wheel chair exiting, and the like. Reducing the incidence of false positives and false negatives would be expected to increase the ability of health care providers to intervene and prevent potentially dangerous falls by the patient.

### SUMMARY OF THE INVENTION

The present invention relates to automated methods and systems for selectively monitoring a patient on a support, such as a bed (e.g., standard hospital bed with side rails), wheelchair, gurney, couch, chair, or recliner, to which the patient may be confined and detecting movements or behaviors that are predictive of support exiting that may lead to a patient fall. The methods and systems are designed to detect and distinguish between movements that are predictive of support exiting and movements that are not.

According to one aspect or embodiment of the invention, one or more cameras, preferably at least two at different angles, can be trained on a patient on a support so as to monitor the position and movements of the patient. The cameras provide a continuous or intermittent, optionally buffered, video stream of the patient to a computer system (e.g., a local computer referred to as the “in room controller”) for continuous or periodic analysis. According to one embodiment, a first camera will be located above the support structure on which the patient rests in order to provide an overhead or bird's eye view of the patient and the support; a second camera will be positioned so as to take a side view of the patient. The overhead camera is able to accurately detect lateral (i.e., side-to-side) and longitudinal (i.e., front-to-back) movements and positioning of the patient; the side camera is able to accurately detect upward and downward, as well as longitudinal, movements and positioning of the patient. A third camera may provide additional functionality, such as a camera mounted adjacent to a video monitor in order to provide direct face-to-face conferencing between the patient and persons outside the patient's room (e.g., staff, other residents, or loved ones). At least one of the cameras may also face a door or other entrance. Absent a triggering event, the video data stream is normally deleted after it has been analyzed by the computer system to preserve patient privacy (i.e., it is typically not archived or viewed by a human).

Using the video data stream provided by the one or more cameras fixed on a patient, the computer system analyzes the position of the patient relative to the support or other reference object (e.g., a wall or proximal stationary equipment) as well as any patient movements in order to determine whether or not the patient is preparing to exit the support. This may be done, for example, by identifying one or more specific patient body parts and measuring their distance from specific parts or locations on the support or other reference object. Changing distances indicate movement. The duration of a limb in a specific position may also be indicative to an intention to support exit (e.g., gripping bedrail by a patient's dominant hand). By way of example but not limitation, in the case where a patient is lying on a typical hospital or extended healthcare facility bed equipped with handrails, one or more of the following body movements or behaviors may be predictive of bed exiting by one patient but not another: (1) sliding down towards the bottom of the bed; (2) right side bedrail roll; (3) left side bedrail roll; (4) torso angle up and leg swing right; (5) torso angle up and leg swing left; (6) torso angle up and upper body roll right; and/or (7) torso angle up and upper body roll left. Similar or alternative positions and movements may be predictive of support exiting when the patient is resting on a different support, examples of which include a wheelchair, gurney, couch, chair, or recliner. If potential support exiting is detected, an appropriate response is triggered, examples of which include alerting staff, communication from the staff to the patient via audio and/or video feeds to the patient's room, prerecorded audio and/or video warnings sent to the patient's room, direct intervention by a staff member, and automated functions, such as bed lowering, raising a bedrail, turning on a light, or actuation of a patient restraint device.

According to one aspect or embodiment of the invention, each patient may have a personal profile that includes, but is not limited to, stored data relating to the specific body movements and behaviors that are most predictive of support exiting by that patient. At least some of the patients may have differing profiles based on specific body movements and behaviors that differ from other patients relative to support exiting. A database of a computer system (e.g., facility master) may be used to compile, store and update patient profiles based on learned information relating to support exiting and other habits of each patient. Utilizing a patient profile that includes data relating to unique body movements and behaviors that have been confirmed to be predictive of support exiting by a particular patient, as compared to general limits and alarms that are universal to all patients, can significantly reduce the incidence of false positives and false negatives compared to conventional systems that do not distinguish between the body movements and behaviors of different patients.

According to another aspect or embodiment of the invention, an information feedback loop may be provided by a system of cameras and monitors to permit human inspection and verification of patient support exiting before initiating audio, visual and/or physical intervention. For example, in the event the computer system detects movements or other behavior predictive of support exiting by a given patient, a video feed of the patient is sent to a monitor at a central station (e.g., nurse's station) subsequent to a visual and/or aural alert to both the nurse's station and the patient's room. An alert is sent to patient's room to warn the patient that someone might be viewing a live video stream of the patient (e.g., by means of a chime, recording, visual display of words, etc.). A staff member (e.g., nurse) can view the view the live video stream from the patient's room to determine if the patient is actually attempting to exit the support. If so, a verification button or other verification means can be actuated by the staff member and appropriate intervention to prevent or assist support exiting is initiated. If not, a reject button or other rejection means can be actuated by the staff member. Intervention may include one or more of establishing an audio/visual link between staff and patient (e.g., in order to stall the patient), sending an alert to proximally located staff for direct physical intervention, and any other known intervention activities. If no response to the alert is given within a prescribe time period, an automated response may be initiated, such as sending a pre-recorded message or warning to the patient and/or alerting nearby staff for direct physical intervention.

The information feedback loop can also be used to update a patient profile to better predict future support exiting. The action of verifying or rejecting an automated support exiting alert based on actual patient movements and behavior can be recorded by the computer system and used to either automatically update the patient profile or provide information for manual updating or adjustment by qualified staff.

When a patient first enters a facility, a general patient profile of common support exiting behaviors may be utilized before specific information is learned regarding a patient's specific support exiting habits. Alternatively, an initial profile is selected from a plurality of standard generic profiles selected and/or initially populated based on information learned by, e.g., the patient filling out a survey and/or or demonstrating support exiting behaviors in the presence of a staff member. As the profile is periodically updated based on verified and/or rejected patient movements and behaviors relative to detected support exiting, it becomes more accurately predictive of actual support exiting of the patient. That reduces the incidence of false positives and false negatives and allows for earlier intervention into the behavior fall sequence. According to one embodiment, patient profiles which have initially coarse granularity due to the lack of known support exiting behaviors may have increasingly fine granularity as the profiles are up. According to one embodiment, patient profiles which have initially coarse granularity due to the lack of known support exiting behaviors may have increasingly fine granularity as the profiles are updated over time to account for learned support exiting behavior. Increasing profile granularity may account for idiosyncratic movements that are entirely unique to a particular patient in addition to the commonly observed movements listed herein.

RFID, ultrasound, or other transmitting devices can be worn or carried by patients, staff and assets to further assist in tracking patient activities, wellness and level of care. A system of sensors may be positioned throughout the facility in order to track the position and movements of each person or asset equipped with a transmitting device. A computer system (e.g., facility master) may continuously record and keep track of the position and time duration at that position of all persons and assets. In the case where a potential support exiting event is detected and verified, an alert for direct physical intervention may be sent to a staff member who is assigned to that particular patient or who is close to the patient and who is not otherwise occupied. The alerted staff member can send verification that intervention was successful. The transmitter (e.g., RFID, ultrasound, etc.) worn by the responder can also be tracked automatically to verify that intervention has occurred.

In the case where a potential support exiting or other triggering event is detected, the video data stream of a patient that may otherwise be deleted upon being analyzed by the computer system may be optionally archived (e.g., recorded on a non-volatile recording medium) for later viewing and analysis of the event. The archived video can be used to confirm that proper protocols and/or prescribed care have been carried out. Events that might trigger video archiving include entry into the patient's room or personal space by staff, patients or visitors, manual alerts or distress signals sent by a patient, detection of other dangerous conditions (e.g., alterations of vital signs or other biometric data), and requested archiving by visiting relatives, friends, doctors or other health care providers.

In addition to or instead of analyzing one or more video streams of the patient resting on a support, other motion detection systems may be employed to monitor the patient and/or patient's room, including one or more of motion sensors, light beams, ultrasound sensors, or RFID sensors. A motion sensor associated with one or more of the cameras can detect motion by, e.g., persons entering the room, person's within the room, and/or the patient.

According to one embodiment, a matrix of light beams and light sensors are positioned laterally and longitudinally above and beside a patient resting on a support (e.g., spaced apart by one-foot intervals). Information concerning the light beams is continuously or periodically monitored to determine potential support exiting. Upon breaking one or more light beams by the patient's body, which is indicative of patient movement and/or body part location, the system can analyze, based on the patient's unique profile of movements that are predictive of support exiting, whether or not the patient is actually attempt to exit the support.

According to another embodiment, a small zone RFID grid or ultrasound grid can be positioned so as to small form RFID zones or ultrasound zones surrounding the patient. In this embodiment, the patient wears several RFID devices or ultrasound devices, such as on the right and left wrists, right and left ankles, and neck, to detect a variety of patient movements and positions simultaneously. The RFID grid or ultrasound grid can include small, closely spaced-apart zones (e.g., one foot square or cubed) that are highly sensitive to even minor movements of the patient. Information concerning the position of the RFID sensors or ultrasound sensors, and by extension the patient, is continuously or periodically monitored to determine potential support exiting. Upon detecting patient movement and/or body part location, the system can analyze, based on the patient's unique profile of movements that are predictive of support exiting, whether or not the patient is actually attempt to exit the support.

The inventive methods and systems for patient monitoring and response described herein may form part of a more comprehensive patient, staff, visitor and asset management method or system at a hospital, nursing home or other healthcare facility. In general, computer controlled methods and systems can be used for maintaining and updating patient data portfolios, locating and assisting patients in need of assistance, preventing or mitigating patient injury, monitoring and archiving video information relating to potentially dangerous activities, and monitoring the location, use and/or activities of assets and personnel as they relate to prescribed activities or treatments.

An integrated system that provides some or all of these features may be provided. An exemplary system includes an interrelated network having some or all of monitoring devices, data storage devices, computing devices, wired and/or wireless data transmission devices, biometric measuring devices, alerting devices, and communication devices. Exemplary monitoring devices include video cameras, radio frequency identification devices (e.g., RFID bracelets and GPS devices), ultrasound devices, and sound monitors (e.g., microphones and speakers). Exemplary data storage and computing devices include devices commonly associated with computer systems. Exemplary alerting devices include alarms, radio transmitters, PDAs, lights, video displays, and speakers. Exemplary communication devices include radio transmitters and receivers, mobile phones, other wireless mobile devices, video displays, and speakers.

According to a first optional enhancement, methods and systems are provided for maintaining, accessing and updating a portfolio of data profiles for multiple patients at a healthcare facility. A database of a computer system (e.g., facility master) includes data that is unique for each patient (i.e., a patient profile). A feedback loop updates each patient profile, as directed by patient and/or staff actions, in order to create and maintain a current database of patient status, attributes and needs. These may include, for example, data relating to patient movements that precede support exiting, patient gait, social interactions, recursive events, prescribed patient care regimens, sound of patient breathing, and patient treatment by movement of, e.g., facility assets and/or personnel. Information relating to a specific patient for input into the computer system may be gathered by means of an RFID and/or ultrasound bracelet worn by the patient that tracks movement, RFID and/or ultrasound devices worn by staff, RFID and/or ultrasound devices on assets used to treat the patient, still shot cameras, video cameras, audio recording devices, etc.

According to a second optional enhancement, a system is provided for locating and aiding a patient in need of immediate assistance. The location of patients or residents can be continuously tracked by means of an RFID bracelet, ultrasound bracelet, or other device worn or carried by each patient that emits a signal that can be detected and traced to a specific location. In one embodiment, the RFID device or ultrasound device also includes an alert device that can be activated in case of emergency of other urgent need. For example, an RFID bracelet or ultrasound bracelet worn by each patient or resident may include a button that, when pushed, can alert an appropriate staff member to provide assistance. Because the RFID bracelet or ultrasound bracelet also provides a means for locating the patient, assistance can be provided quickly and efficiently even if the patient cannot communicate. According to one embodiment, two-way audio-visual communication may be initiated via a camera, video monitor, microphone and speaker. The alerting system may optionally access patient-specific information stored for the patient wearing a particular RFID bracelet or ultrasound bracelet who sent the alert in order to tailor the response to the specific needs of that patient. Patient usage of the alert feature can be tracked, analyzed and used to update patient profiles.

According to a third optional enhancement, selective archiving of otherwise volatile video recordings of a patient may be actuated in response to a potentially dangerous or other pre-selected triggering event. In general, it may be desirable to continuously monitor or record a patient while in the patient's room (e.g., to predict and mitigate injury that may result from unassisted actions, such as support exiting), while not permanently archiving or saving the video data stream. Thus, while the video stream may be temporarily buffered for analysis by the computer system, it is typically erased or deleted on an ongoing basis and not viewed by human eyes in order to protect patient privacy. This is the default scenario assuming no triggering event. Examples of triggering events include attempted support exiting or other potentially dangerous patient actions or behaviors, entry by authorized staff wearing properly encoded RFID bracelet or ultrasound bracelet to document existence or absence of proper care, entry by unauthorized staff or other person not wearing a properly encoded RFID bracelet or ultrasound bracelet (optionally coupled with an alert to responsible staff to remove the unauthorized person), and the presence in a patient's room of an asset equipped with properly encoded RFID device or ultrasound device. Video archiving can be used to validate a healthcare facility by documenting that proper care was being provided at the time a potentially dangerous event may have occurred.

According to a fourth optional enhancement, the locations and movements of staff, assets, patients and third parties can be monitored to determine the sufficiency of activities and limits. For example, it may be incumbent for one or more of a staff member, asset or patient to be in a prescribed location. By monitoring the positions and time durations at those positions of various persons or equipment, it can be determined whether or not the prescribed activity was carried out and, if so, whether it was carried out properly. At the other end of the spectrum are prescribed limits. For example, different patients, staff or visitors may have varying and unique limits on how much of the facility they are authorized to access. When unauthorized patient wandering is detected (e.g., by specially encoded RFID devices, ultrasound devices, and/or GPS devices worn or carried by the patient), staff can be alerted to prevent or mitigate such wandering. When unauthorized entry into forbidden zones by patients, visitors or unauthorized staff is detected, staff can be alerted to prevent or mitigate such unauthorized entry. The method can also be used to chronicle wellness events, such as social interactions involving face-to-face encounters and/or video conferencing involving a patient, another patient, a visitor, a health care providers, staff, etc.

Information relating to any event that relates to any aspect of the invention can be analyzed, stored or otherwise processed by a computer system. Each healthcare facility may have a centralized computer system, referred to as the “facility master.” The facility may also have resident location computer systems that are networked with the facility master (e.g., in each patient room (i.e., “in room controller”), staff station, and the like). Any of the inventive processes can be implemented by a computer system. The computer system may comprise one or more computer-readable media (e.g., physical storage devices such as hard drives, memory devices, magnetic tapes or disks, optical storage media, or other known digital storage device) that contain executable instructions for carrying out the computer-implemented aspects of the inventive methods and systems.

These and other advantages and features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 schematically illustrates an exemplary system for patient monitoring, alert and response;

FIGS. 2A-2D schematically illustrate exemplary configurations of patient rooms at a healthcare facility equipped for patient monitoring and response to support exiting;

FIG. 3 is a flow chart that illustrates an exemplary method for monitoring a patient on a support, detecting possible support exiting, and initiating a response to prevent or mitigate patient harm;

FIGS. 4A-4E schematically depict a patient in various exemplary positions on a bed relative to known bed exiting behaviors;

FIG. 5 is a flow chart that illustrates an exemplary computer-controlled method for determining if a patient is preparing to exit a support within a predetermined coordinate space;

FIG. 6 is a flow chart that illustrates an exemplary method for generating and updating a patient profile that contains data relating to support exiting behavior of that patient;

FIG. 7 is a flow chart that illustrates an exemplary method for responding to a computer predicted support exiting event;

FIG. 8 is a decision chart that illustrates an exemplary decision sequence for responding to an alert of predicted bed exiting;

FIG. 9 schematically illustrates exemplary computer architecture that facilitates facility, patient, staff and/or asset monitoring and event response management;

FIG. 10 is a flow chart that illustrates an exemplary method for managing a response to an actionable event in a healthcare facility;

FIG. 11 is a flow chart that illustrates an exemplary method for maintaining alarm levels in a patient risk profile for a patient of a healthcare facility;

FIG. 12 schematically illustrates various exemplary network protocols that can be used to facilitate communication between computer systems in a healthcare facility;

FIG. 13 schematically illustrates an exemplary facility monitoring master system;

FIG. 14 is a flow chart that illustrates an exemplary method for maintaining stored profiles for a plurality of patients at a healthcare facility;

FIG. 15 is a flow chart that illustrates an exemplary method for providing an automated response to a patient initiated alert;

FIG. 16 is a flow chart that illustrates an exemplary method for selective archiving of a video data stream of a patient in response to a triggering event; and

FIG. 17 is a flow chart that illustrates an exemplary method for monitoring the adequacy of patient health and/or wellness based on the patient's personal profile.

### DETAILED DESCRIPTION OF THE PREFERRED EMBODIMENTS

I. Introduction

Embodiments of the present invention extend to methods, systems, and computer program products for managing patient care and wellness at a healthcare facility. The invention more particularly relates to computer-controlled methods and systems for monitoring a patient on a support such as a bed (e.g., standard hospital bed with side rails), wheelchair, gurney, couch, chair, or recliner to which the patient may be confined and detecting a position, movement or behavior that is predictive of support exiting that may lead to a patient fall. The methods and systems are designed to detect and distinguish between movements that are predictive of support exiting and movements that are not.

The term “patient fall” shall be broadly understood to include falling to the ground or floor, falling into stationary or moving objects, falling back onto a support, or any other falling motion caused at least in part by gravity that may potentially cause physical injury and/or mental or emotional trauma.

The terms “rest” and “resting” as it relates to a patient resting on a support shall be broadly understood as any situation where the support provides at least some counter action to the force of gravity. Thus, a patient may “rest” on a support while lying still, sitting up, moving, lying down, or otherwise positioned relative to the support so long as the support acts in some way to separate a patient from the floor or surface upon which the support is itself positioned.

The terms “continuous monitoring” and “continuous video data stream” include taking a series of images that may be spaced apart by any appropriate time interval so long as the time interval is sufficiently short that the system is not unduly hampered from initiating a response in time to prevent or mitigate a potentially dangerous event.

The terms “receiving” and “inputting” in the context of a patient profile broadly includes any action by which a complete or partial patient profile, or any component thereof, is stored or entered into a computer system. This includes, but is not limited to, creating a profile and then storing or entering it into a computer, entering data which is used by the computer to generate a new patient profile, and/or storing or entering data used by a computer for updating a pre-existing patient profile already in the computer.

The inventive methods and systems for monitoring a patient on a support and initiating an alert to prevent or mitigate harm in the event of support exiting may stand on their own or form part of a more comprehensive patient monitoring and wellness system. Generally, wireless monitoring and safety management systems identify, store, transmit, and report on data related to patients, providers, events, and equipment in a healthcare facility. Data can relate to patient movements, patient entering and exiting furniture or other supports, patient initiated manual alarms, automated alarms, patient schedules, and patient care instructions. Alarm, alert, and activity responses utilize a wired and/or wireless network to automatically provide real-time two-way communication between one or more providers and one or more patients, to escalate activities, to log data for later interpretation, and to audit completion of activities related to patient care.

When specific behavior and/or actions are detected within a healthcare facility, an alarm or alert can be sent to one or more appropriate providers for event verification and/or direct intervention. Alarms and alerts can be sent to a central station, to personal digital assistants (or other mobile computer systems), wireless tracking devices, to audio/video alarm systems or to other medical, security, and management information systems. Automated responses can attempt to delay a patient's detrimental activity (e.g., unattended bed exiting) until a provider can move to the patient's location to intervene.

Patient specific data can be collected for each patient to create a database of generalized and personalized knowledge. Healthcare facilities and providers can use the database of knowledge to better understand risks associated with various activities for each patient and/or for each type of activity. Predictive modeling and artificial intelligence can be applied to collected data patterns to identify, process, categorize, alarm, and rectify risks based on patient information, such as, for example, patient type, patient activity, patient medications, patient physical therapy process, patient location, and other variables.

Patient profiles can be maintained for each patient based on corresponding patient specific data. Patient profiles can be iteratively refined as additional patient specific data is collected. From a patient profile, meaningful and appropriate alarm levels can be configured for a patient to provide the patient with increased and/or more personalized care, safety and security. Other automated modules can manage patient schedules, medications (dosages, route of administration, time for administration, etc.), physical therapy schedule, activity appointments, physician orders, and other patient care information. In this way, general patient health, safety and wellness can be automatically monitored.

Those skilled in the art will appreciate that the invention may be practiced in network computing environments with many types of computer system and electronic device configurations, including, personal computers, desktop computers, laptop computers, hand-held devices, multi-processor systems, microprocessor-based or programmable consumer electronics, network PCs, minicomputers, mainframe computers, mobile telephones, PDAs, one-way and two-way pagers, Radio Frequency Identification (“RFID”) devices (e.g., bracelets, tags, etc.), ultrasound devices (e.g., bracelets, tags, etc.), global position (“GPS”) devices, and the like. The invention may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present invention may comprise or utilize a special purpose or general-purpose computer including computer hardware, as discussed in greater detail below. Embodiments within the scope of the present invention also include physical and other computer-readable media for carrying or having computer-executable instructions or data structures stored thereon. Such computer-readable media can be any available media that can be accessed by a general purpose or special purpose computer system. Computer-readable media that store computer-executable instructions are physical storage media. Computer-readable media that carry computer-executable instructions are transmission media. Thus, by way of example, and not limitation, computer-readable media can comprise physical storage media or transmission media.

Physical storage media, such as, RAM, ROM, EEPROM, CD-ROM or other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can comprise a network or data links which can be used to carry or store desired program code means in the form of computer-executable instructions or data structures and which can be accessed by a general purpose or special purpose computer. Combinations of the above should also be included within the scope of computer-readable media.

Further, it should be understood, that upon reaching various computer system components program code means in the form of computer-executable instructions or data structures can be transferred automatically from transmission media to physical storage media. For example, computer-executable instructions or data structures received over a network or data link can be buffered in RAM within a network interface card, and then eventually transferred to computer system RAM and/or to less volatile physical storage media at a computer system. Thus, it should be understood that physical storage media can be included in computer system components that also (or even primarily) utilize transmission media.

Computer-executable instructions comprise, for example, instructions and data which cause a general purpose computer, special purpose computer, or special purpose processing device to perform a certain function or group of functions. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

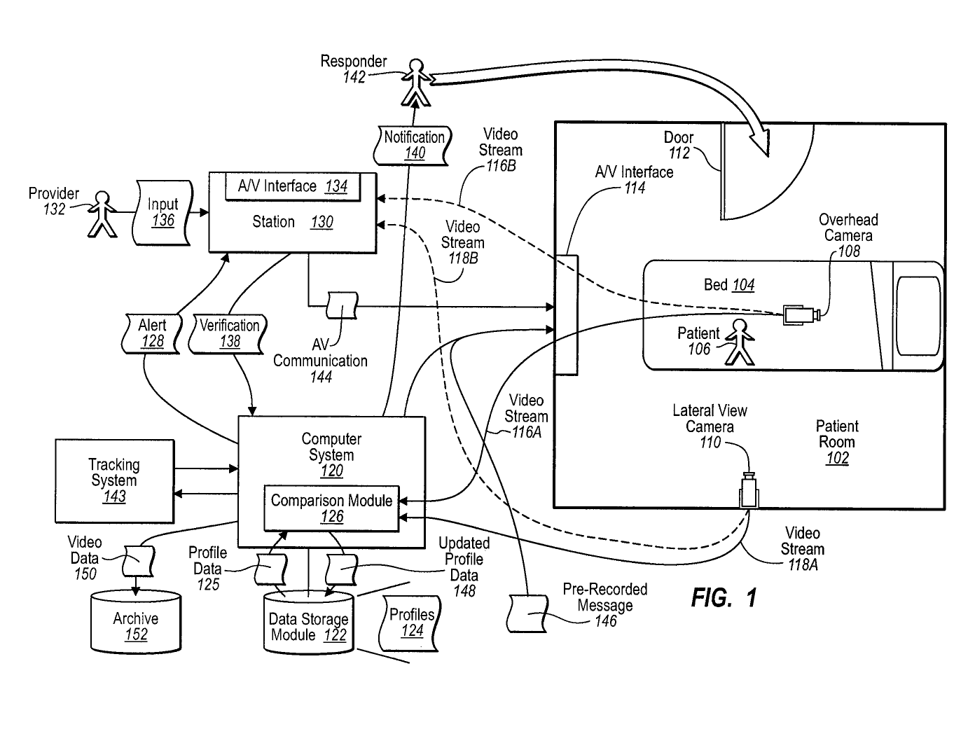

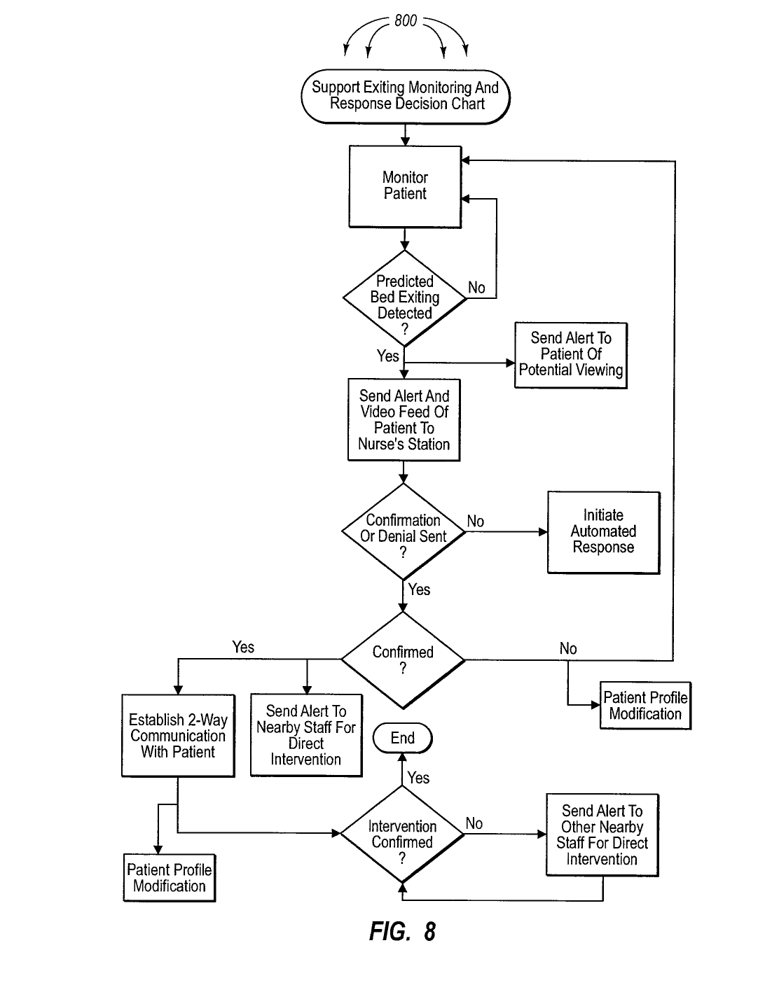

FIGS. 1-7 and the accompanying description below illustrate exemplary methods and systems for monitoring a patient on a support and initiating an appropriate response in order to prevent or mitigate harm to the patient in the event of actual or predicted support exiting. FIGS. 8-12 and the accompanying description below illustrate exemplary systems and methods for generalized and specific patient, caregiver, visitor and asset monitoring, care and response. FIGS. 13-16 and the accompanying description below illustrate exemplary optional enhancements to the inventive methods and systems of the present invention.

II. Systems and Methods for Monitoring Patient Support Exiting and Response

FIG. 1 is a diagram that schematically illustrates an exemplary computer-controlled system 101 for patient monitoring, more particularly with respect to monitoring potential support exiting, detecting a position and/or movement of a patient that is predictive of support exiting, obtaining human verification of actual support exiting, and intervening if support exiting is confirmed. The patient monitoring system 101 includes a patient room 102 containing a bed 104 or other support and a patient 106 resting thereon at least some of the time. One or more overhead cameras 108 may be provided that provide an aerial view of patient 106 together with one or more side cameras 110. The overhead camera 108 is especially useful in monitoring lateral (i.e., side-to-side) and longitudinal (i.e., head-to-foot) patient movements, although it may also monitor other movements. The side camera 110 is especially useful in monitoring longitudinal and up and down movements, although it can monitor other movements. The side camera or other camera (not shown) can be positioned to monitor and record a patient room door 112 or other access point (e.g., to record entry and/or exit of personnel, other patients, and visitors). The bed 104 may include markings (e.g., decals) (not shown) that assist in properly orienting the cameras.

The room 102 also includes an audio-video interface 114 that can be used to initiate one-way and/or two-communication with the patient 106. A/V interface 114 may include any combination of known A/V devices, e.g., microphone, speaker, camera and/or video monitor. According to one currently preferred embodiment, A/V interface 114 is mounted to a wall or ceiling so as to be seen by patient 106 (e.g., facing the patient's face, such as beyond the foot of the patient's bed). The A/V interface 114 includes a video monitor (e.g., flat panel screen), a camera mounted adjacent to the video monitor (e.g., below), one or more microphones, and one or more speakers. The A/V interface may form part of a local computer system (e.g., an “in room controller”) that controls the various communication devices located in the patient room.

In order to analyze patient movements that may be predictive of support (e.g., bed) exiting, video data streams 116A and 118A are sent from cameras 108 and 110, respectively, to a computer system 120 for analysis. According to one currently preferred embodiment, at least a portion of the computer system 120 is an in room controller associated with the patient room 102. In the case where each patient room has its own in room controller, patient monitoring and analysis can be performed in parallel by dedicated in room controller computers. Nevertheless, at least some of the tasks, information, and information flow may be performed by a remote computer, such as a central facility master computer. The computer system 120 may therefore include multiple networked computers, such an in room controller, facility master, and other remote computers. The computer system 120 includes or has access to a data storage module 122 that includes patient profiles 124 (e.g., stored and updated centrally in the facility master and used locally by and/or uploaded to the in room controller).

A comparison module 126 of the computer system 120 analyzes the video streams 116A, 118A and, using one or more algorithms (e.g., that may be known in the art or that may be developed specifically for this system), determines the location and/or any movements of patient 106. This information is compared to patient specific profile data 125 from a patient profile 124 that corresponds to patient 106. In the absence of predicted support exiting or other triggering event, video streams 116A and 118A are typically not viewed by any human but are deleted or simply not stored or archived. This helps protect patient privacy.

When a location and/or movement of patient 106 matches or correlates with profile data 125 predictive of support exiting by patient 106, the computer system 120 sends an alert 128 to a central station 130 (e.g., nurse's station) that patient 106 may be attempting to exit support 104. In addition to the alert 128, at least one of video streams 116B and 118B from cameras 108 and 110 and/or a modified video stream (not shown) from computer system 120 is sent to an A/V interface 134 at central station 130 for human verification of actual patient support exiting. The patient 106 is advantageously notified of potential active viewing by staff to satisfy HIPAA regulations (e.g., by a chime, prerecorded message, e.g., “camera is actively viewing”, or visual indication, e.g., flashing or illuminated words, TV raster pattern). A provider 132 views the video stream(s) from patient room 102, determines whether the patient 106 is in fact preparing to exit the bed 104 or other support, and provides verification input 136 to an appropriate interface device (not shown) at station 130, which sends verification 138 to the computer system 120. Verification 138 may either confirm or reject the determination of patient support exiting. When viewing is terminated, the patient may be notified of this fact by, e.g., a tone or pre-recorded message (“active viewing is terminated”).

If the provider 132 determines and verifies that actual patient support exiting is occurring or about to occur, the in room controller, facility master, or other appropriate module or subsystem component within computer system 120 sends a notification 140 to a responder 142 to assist the patient 106. Notification 140 may be sent by any appropriate means, including an audio alert using a PA system, a text and/or audio message sent to a personal device carried by responder 142, a telephone alert, and the like. A tracking system 143 that interfaces or communicates with the computer system 120 (e.g., the facility master) may be used to identify a caregiver 142 who is assigned to patient 106 and/or who is nearest to patient room 102. In this way, direct physical assistance to patient 106 who may be attempting to exit support 104 can be provided quickly and efficiently.

In addition to or instead of sending notification 140 to responder 142, one- or two-way A/V communication 144 can be established between provider 132 at central station 130 and patient 106 (e.g., by means of A/V interfaces 114 and 134). This allows provider 132 to talk to patient 106 in order to provide instructions or warnings regarding support exiting, possibly to distract patient 106 and delay or prevent support exiting (e.g., “why are you getting out of bed?”). This may allow responder 142 to more easily intervene prior to actual support exiting so as to prevent or better mitigate potential harm to patient 106. A pre-recorded audio and/or A/V message 146 may alternatively be sent to A/V interface 114 in patient room 102 instead of direct A/V communication between provider 132 and patient 106.

In the event a provider 132 is not present at central station 130 or otherwise fails to provide verification 138 regarding predicted support exiting within a prescribed time period, the computer system 120 may initiate an automated response in order to prevent or mitigate potential harm to patient 106. This may include one or both of sending notification 140 to a responder 142 regarding possible support exiting and/or sending a pre-recorded message 146.

Verification 138, whether confirmation or denial of actual support exiting, can also be used to update the patient profile 124 corresponding to patient 106. Updated profile data 148 based on one or more support exiting events can be input or stored at data storage module 122. If a particular behavior is found to accurately predict support exiting by patient 106, the patient profile 124 can be updated to confirm the accuracy of the initial profile 124. In some cases, limits within the patient profile 124 may be tightened to be more sensitive to movements that have been confirmed to correlate with and accurately predict support exiting. This may be done manually by authorized personnel or automatically by the computer system 120. If, on the other hand, a particular behavior is determined to falsely predict support exiting by patient 106, the patient profile can be updated to note incidences of such false positives Limits within the patient profile 124 can then be loosened or eliminated relative to any movements that have been found not to correlate with support exiting by patient 106. In the event support exiting by patient 106 occurs but is not detected by the computer 120, limits within the patient profile 124 can be established and/or tightened in an effort to eliminate false negatives of support exiting by patient 106. Updating the profile 124 of patient 106 to more accurately predict support exiting and reduce or eliminate false positive and false negatives substantially increases the reliability of the patient monitoring system as compared to conventional systems that do not distinguish between and among support exiting habits or behaviors of different patients.

In order to later view and/or analyze a triggering event as may be established by a facility, video data 150 that is the same as, or which may be derived from, one or both of video streams 116 and 118 can be stored within an archive 152. Archive 152 may comprise any storage media known in the art of video recording and storage, examples of which include hard drives, optical storage devices, magnetic tapes, memory devices, and the like.

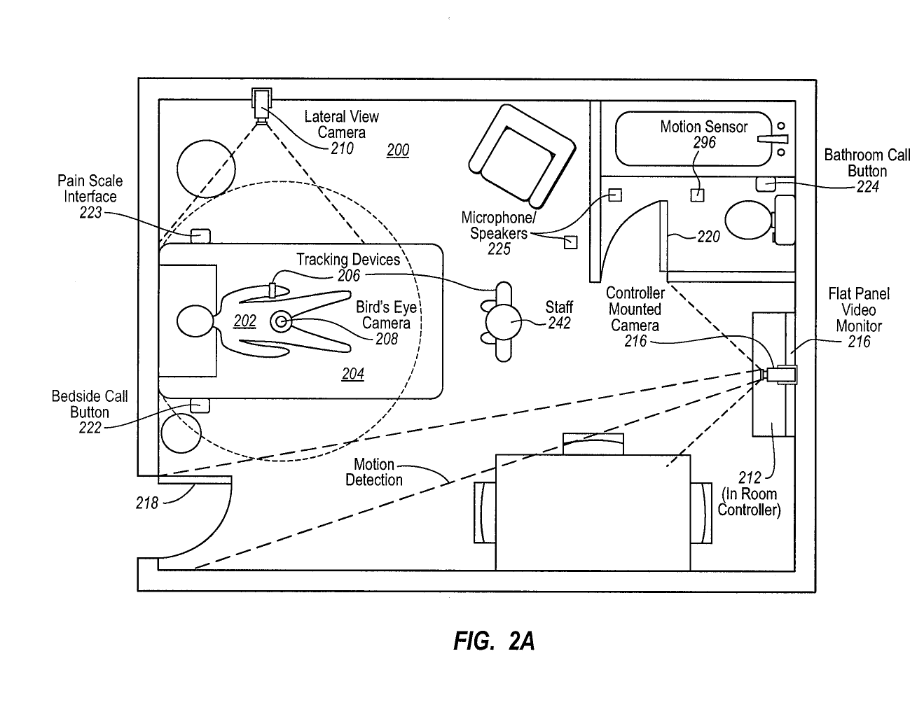

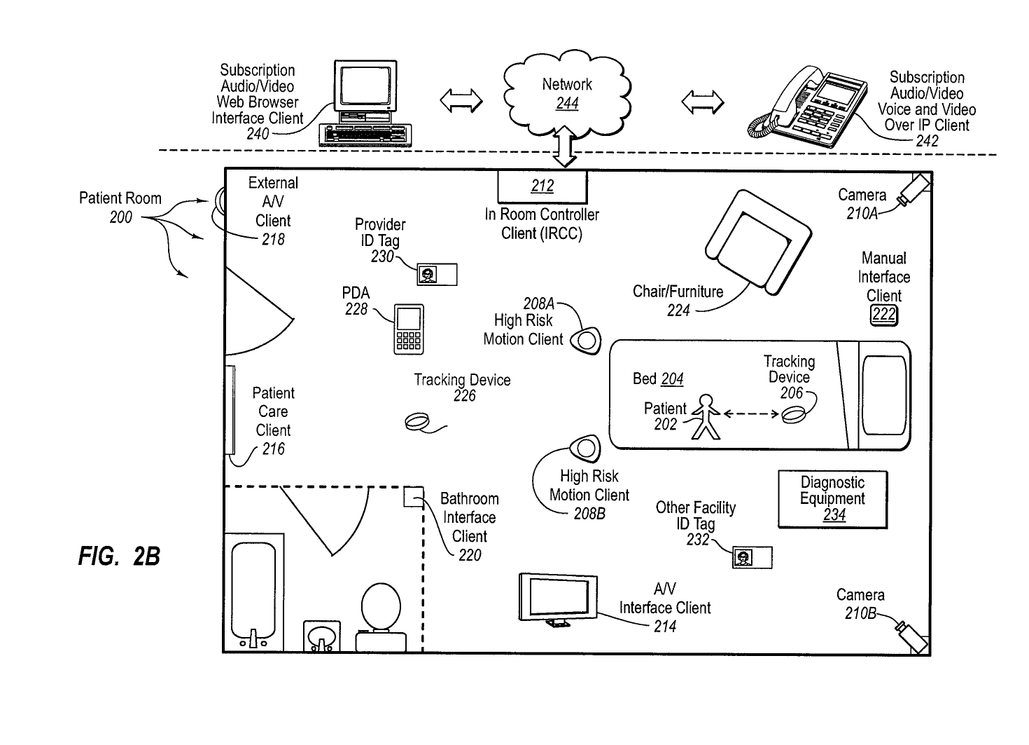

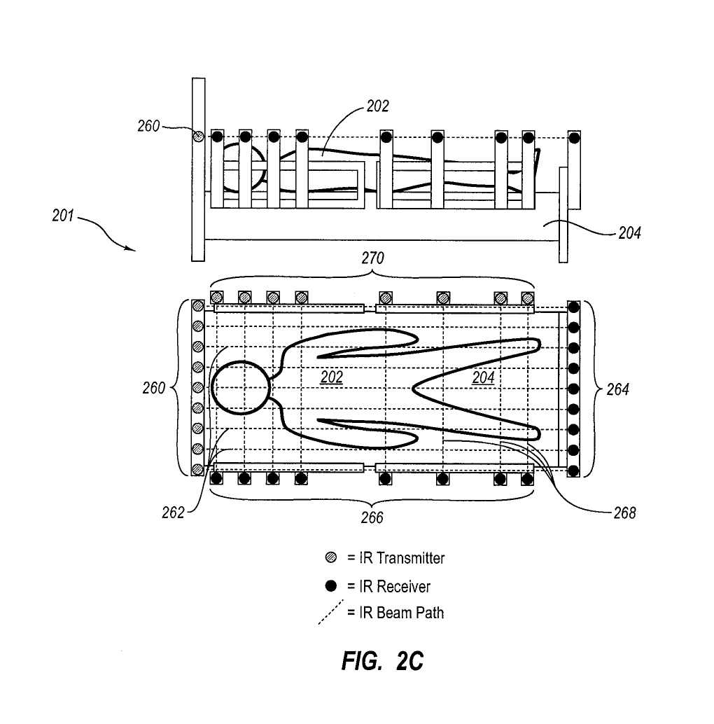

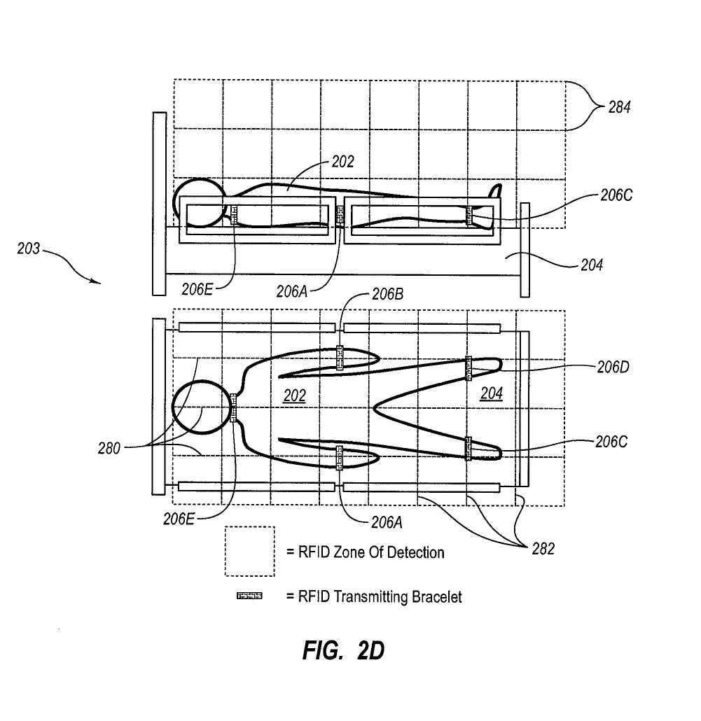

FIGS. 2A-2D schematically illustrate various embodiments of exemplary patient room configurations used in monitoring a patient and providing one or more responses. In the embodiment of FIG. 2A, an exemplary patient room 200 is illustrated which includes a patient 202, a bed 204 or other support upon which the patient 202 rests at least some of the time. The patient 202 may wear or carry a mobile electronic tracking device 206, such as an RFID bracelet, ultrasound bracelet, or other device. This allows a facility master computer to identify and track the location of the patient 202 by means of electronic tracking systems known in the art. Device 206 is specially assigned to patient 202 and provides verification when patient 202 is located in room 200. This facilitates using the correct patient profile when interpreting movements of patient 202 rather than those of another patient.

One or more overhead cameras 208 are positioned above the bed 204 and so as to provide an aerial (e.g., bird's eye) view of patient 202. One more side cameras 210 are positioned to the side of patient 202 to provide a different data stream for determining the patient's position and/or movements. Camera 210 may have a direct or peripheral view of a door 218 or other entrance to room 200. An in room controller computer (IRCC) 212, which may be a local computer located in room 200, at least partially controls and is in communication with cameras 208, 210. A flat panel monitor 214 (e.g., high definition), controller mounted camera 216, and optionally other devices such as microphones and speakers (not shown) are interfaced with IRCC 212.

The IRCC 212 is used to determine the location of the patients body, including specific body parts, by interpreting video data streams generated by one or more of the cameras and comparing relative distances between the patient's body and fixed locations (e.g., the patient's head and the headboard of the bed, the patient's arms and legs relative to the bedrails, the height of the patient's torso relative to the bed, etc.). A changing body part position indicates movement of that body part. The IRCC 212 continuously or periodically compares the location and/or any movements of the patient's body or portion thereof with locations and movements predictive of patient bed exiting by that patient as contained in the patient's profile of bed exiting behaviors. Whenever a position and/or movement is detected that is consistent with bed exiting, an appropriate response is initiated as discussed elsewhere.

The flat panel video monitor 214 can provide multiple functions, including providing normal television programming, recorded programming requested by the patient 202, video feeds remote locations (such as loved ones and staff who wish to communicate with patient 202 remotely), and special messages (e.g., patient alerts). The controller mounted camera 216 provides a direct facial view of the patient and, in combination with video monitor 214, facilitates two-way A/V communication between patient 202 and person's outside room 200. As shown, the camera 216 may also have a direct view of a door 218 or other entrance to monitor entry and exit of persons (e.g., staff 242) from room 200. Camera 216 may also have a view of bathroom door 220 to monitor movement of patient 202 to and from the bathroom. A standard motion sensor integrated with conventional video cameras (e.g., camera 216) may provide motion detection means for monitoring room entry or exiting activity.

The room 200 may include other auxiliary devices, such as bedside call button 222, bedside patient pain scale interface 223, bathroom call button 224, microphones/speakers 225, and bathroom motion sensor 296. Call buttons are known in the art. The pain scale interface 223 allows a patient to indicate to the monitoring system (e.g., IRCC 212, facility master, and/or nurse's station) the patient's current pain level (e.g., on a scale of 1 to 10, with 1 being the least and 10 being the most pain). Motion sensor 296 can be used, e.g., in combination with camera 216, call button 224 and/or microphones/speakers, to determine whether a patient 202 requires further assistance while in the bathroom. An RFID grid set up throughout the room can be used to monitor the position and/or movements of the patient 202 when not resting on the bed 204, as well as the position and/or movements of staff 242, other persons such as patients, friends, family or other visitors, and assets (not shown).

FIG. 2B illustrates an exemplary patient room 200 which includes a patient 202, a bed 204 or other support upon which the patient 202 rests at least some of the time, and various other devices used to monitor the patient and the patient's room 200. The patient 202 may wear or carry a mobile electronic tracking device 206. This allows a facility master computer to identify and track the location of the patient 202 by means of electronic tracking systems known in the art. Tracking device 206 may be a conventional RFID device or ultrasound device (e.g., bracelet) and may be equipped with a patient call or panic button (not shown) as known in the art. Tracking device 206 is specially assigned (and attached) to patient 202 staying in patient room 200. Tracking device 206 provides verification that patient 202 is actually located in room 200. This facilitates using the correct patient profile when interpreting movements of patient 202 rather than those of another patient.

High risk motion clients 208A and 208B (e.g., which include one or more of cameras, electronic motion sensors, electric eyes, RFID detectors, ultrasound detectors, etc.) may be positioned on either side of bed 204, thus providing two separate data streams for interpretation of the patient's position and/or movements. Side cameras 210A and 210B are positioned on either side of patient 202 to provide additional data streams for interpretation of the patient's position and/or movements. At least one of cameras 210A and 210B may have a direct or peripheral view of a door 211 or other entrance to room 200. An in room controller client (IRCC) 212, which can be a local computer located in or near room 200, at least partially controls motion clients 208A and 208B, cameras 210A and 210B, and other electronic devices in room 200. IRCC 212 also analyzes video data generated by cameras 208, 210 in order to identify behavior of patient 202 that may be predictive of support exiting.

Other electronic devices include an in-room ANV interface client 214, which can be used to establish one- or two-way communication with patient 202, patient care client 216, external ANV client 218 (e.g., in a hallway), bathroom interface 220 (e.g., call button, microphone and/or speaker), and manual patient interface client 222 (e.g., a call button, pain scale dial, etc.). The room is shown having a chair 224 or other furniture (e.g., wheel chair), upon which visitors or even the patient may rest at least some of the time. The monitoring system can be used to detect potential support exiting by patient 202 of chair/furniture 224 in addition to bed 204.

The IRCC 212 and electronic devices in room 200 can interoperate to implement the principles of the present invention. High risk motion clients 208A and 208B, either alone or in combination with one or both of cameras 210A and 210B, can monitor a patient's movements in bed 204 and/or chair or other furniture 224. Generally, a patient's movement on a bed or other support can be monitored through a grid monitoring system (“GMS”) that identifies patient vertical and horizontal movements that may be indicative of an attempt to exit the furniture. The time a body part is located within a critical zone and/or changes in position and/or changes in speed can all be determined. The GMS can also utilize pressure, temperature, and other distributed sensors located within a bed or other furniture or directly attached to a patient. Inputs from the various clients and sensors in room 200 can be provided to the IRCC 212 and/or facility master (not shown). In addition, any of cameras 210A, 210B or 220, as well as motion clients 208A and 208B, can monitor a patient's position and/or movements within room 200 when the patient is not resting on a bed 204, chair 224 or other support located in room 200.

Upon activation of the GMS or other high risk motions clients, in room controller client 212 and/or a facility master utilizes patient management software to initiate and establish responsive actions. For example, upon detecting activities that predict an unattended support exit, in room controller 212 and/or a facility master can establish a real time AN connection with a central station (e.g., nurse's) and/or one or more mobile caregiver clients (e.g., PDAs carried by responder caregivers). Further, in room controller client 212 and/or a facility master can activate external A/V client 218 (e.g., an alarm in a hallway) and/or initiate archiving of data from one or more of high risk motion clients 208A and 208B, and cameras 210A, 210B and 220 upon the occurrence of a support exiting event or other pre-established triggering event.

FIG. 2B further depicts a provider tracking device 226 (e.g., an RFID or ultrasound device), a provider PDA 228, a provider ID tag 230 (e.g., an RFID or ultrasound device), other facility ID tag 232 (e.g., an RFID or ultrasound device), and/or diagnostic equipment 234 which have entered room 200. Each of these devices can communicate with IRCC 212 and/or a system-wide tracking system that communicates direct to a facility master computer (not shown) via various appropriate protocols (e.g., RF, ultrasound waves, IEEE 802.11 group, IEEE 802.15.4, etc.). IRCC 212 can update pertinent patient information, such as, for example, provider ID, other personnel ID or diagnostic equipment and time of entry. Detecting the presence of personnel and devices inside room 200 indicates that facility personnel and/or assets associated with these devices have likely entered room 200, for example, in response to a predicted support exiting event, a patient initiated alarm, prescribed patient activities, and the like.

According to one embodiment, patient room 200 may be networked with other components including, for example, subscription clients 240 and 242 (e.g., subscription A/V web browser interface client 240 and subscription A/V voice and video over IP client 242), which are connected to in room controller client 212 by means of network 244. Subscriber clients 240 and 242 can be located at or external to a healthcare facility. Thus, providers in diverse locations can be notified of actionable events occurring inside patient room 200.

FIG. 2C illustrates an alternative embodiment for detecting patient support exiting behavior comprising a light beam matrix system 201, which may be used instead of or in addition to one or more cameras used to determine patient position and/or movements. Exemplary light beam matrix system 201 includes a patient 202 resting on a bed 204 or other support. A plurality of light transmitters 260 are positioned at one side of bed or other support 204 and generate first beams of light 262, which are detected by corresponding first light receivers 264. A plurality of second light transmitters 266 are positioned laterally relative to first light transmitters 260 and generate second beams of light 268, which are detected by corresponding second light receivers 270. Beams of light 262, 268 may comprise IR, visible or UV wavelengths.

First and second beams of light 262, 268 may be positioned above the patient 202 and cross-cross to form a light beam matrix that is able to detect patient location and/or movement in multiple (e.g., three) dimensions. The closer together the light beams, the finer the detection of patient position and/or movement. According to one embodiment, the light beams are spaced apart at intervals ranging from 6 inches to 2 feet (e.g., at 1 foot intervals). As long as the patient 202 rests flat on the bed or other support 204 or is otherwise below the light beam matrix comprising first and second light beams 262, 268, no beams of light are blocked or interrupted such that no movement is detected. Interrupting and/or resuming one or more beams of light may be indicative up upward and/or downward movement(s). Sequentially interrupting and/or resuming one or more of first light beams 262 may be indicative of lateral movement(s). Sequentially interrupting and/or resuming one or more of second light beams 262 may be indicative of longitudinal movement(s).

A computer system (not shown) interprets data generated by the light beam matrix. Continuous light detection by the light sensors may be interpreted as a series of 1s (or 0s) in computer language. Any interruption or blocking of a light beam corresponds to a series of 0s (or 1s) in computer language and is indicative of a body part being positioned between one or more light particular light transmitters and detectors. Because bed exiting, for example, involves at least some lifting of the patient's body (e.g., to get over bed rails or pass through a narrow passage in a bed rail), actual lifting of the patient's body will typically block or interrupt at least one light beam. Depending on which light beams are interrupted, the computer can determine which parts of the patient's body have raised and/or moved. Crossing multiple beams typically indicates movement (i.e., lateral, longitudinal, upward and/or downward depending on which sequence of beams are interrupted). The patient's movements, as detected by the light beam matrix and interpreted by the computer system, are compared to a patient profile of positions and/or movements that are predictive of support exiting by that patient. If potential patient support exiting is detected, an appropriate response can be initiated.

FIG. 2D illustrates an alternative embodiment for detecting patient support exiting behavior comprising a small zone RFID grid system 203, which may be used instead of or in addition to one or more cameras used to determine patient position and/or movements. Exemplary RFID grid system 203 includes a patient 202 resting on a bed 204 or other support. The patient's body may be equipped with any appropriate number of RFID devices that are located so as to detect patient positions and/or movements associated with support exiting (e.g., right RFID wrist device 206A, left RFID wrist device 206B, right RFID ankle device 206C, left RFID ankle device 206D, and neck RFID device 206E). Each RFID device can be separately encoded to represent a specific body part of the patient to distinguish between positions and movements of the different body parts.

The RFID grid system 203 includes a three-dimensional grid of small, cube-like RFID zones defined by a plurality of RFID detectors positioned along lateral zone boundaries 280, longitudinal zone boundaries 282, and elevation zone boundaries 284. The closer together the RFID detectors, the finer the detection of patient position and/or movement. According to one embodiment, the RFID detectors are spaced apart at intervals ranging from 6 inches to 2 feet (e.g., at 1 foot intervals). The grid of RFID zones is able to detect three-dimensional patient position and/or movements as approximated by the positions and/or movements of the RFID devices 206 worn by the patient in or through the RFID zones.

A computer system (not shown) interprets data generated by the small zone RFID grid as it detects the position and/or movement of the RFID devices 206 attached to the patient 202. Depending on which RFID zone is occupied by a specific RFID device and/or which RFID device(s) may be moving between RFID zones, the computer can determine the position and/or location of corresponding body parts of the patient. If potential patient support exiting is detected, an appropriate response can be initiated.

A similarly configured ultrasound grid system can also be used to implement the functionality depicted in FIG. 2D. A patient's body may be equipped with any appropriate number of ultrasound devices that are located so as to detect patient positions and/or movements associated with support exiting. Each ultrasound device can be separately encoded to represent a specific body part of the patient to distinguish between positions and movements of the different body parts.

Thus, an ultrasound grid system can also include a three-dimensional grid of small, cube-like ultrasound zones defined by a plurality of Utlrasound detectors positioned along lateral zone boundaries 280, longitudinal zone boundaries 282, and elevation zone boundaries 284. The closer together the ultrasound detectors, the finer the detection of patient position and/or movement. According to one embodiment, the ultrasound detectors are spaced apart at intervals ranging from six (6) inches to two (2) feet (e.g., at one (1) foot intervals). The grid of ultrasound zones is able to detect three-dimensional patient position and/or movements as approximated by the positions and/or movements of the ultrasound devices worn by the patient in or through the ultrasound zones.

Accordingly, a computer system (not shown) can interpret data generated by the small zone ultrasound grid as it detects the position and/or movement of the ultrasound devices attached to the patient 202. Depending on which ultrasound zone is occupied by a specific ultrasound device and/or which ultrasound device(s) may be moving between ultrasound zones, the computer can determine the position and/or location of corresponding body parts of the patient. If potential patient support exiting is detected, an appropriate response can be initiated.

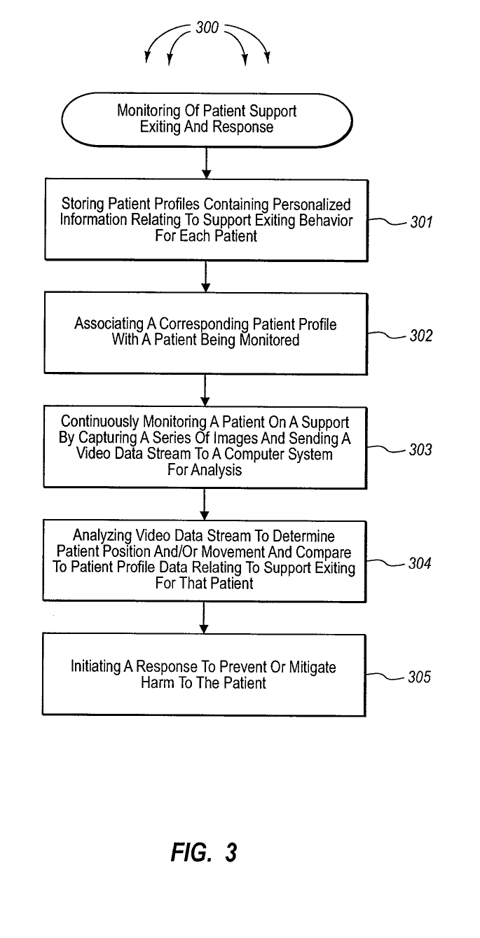

FIG. 3 is a flow chart that schematically illustrates an exemplary method 300 of monitoring a patient in order to detect support exiting and initiate a response in the event of predicted support exiting. This method may be carried out at least in part using the exemplary patient monitoring systems illustrated in FIGS. 1-2C discussed above and/or systems illustrated or discussed elsewhere in this disclosure and/or systems or components known in the art. A first step 301 involves creating or obtaining a plurality of patient profiles, each containing personalized information relating to support exiting behavior for each patient.

Examples of known bed exiting behaviors that have been observed as being used by one or more patients include, but are not limited to: (1) bed slide method (e.g., sliding down towards the bottom of the bed); (2) right side rail roll method; (3) left side rail roll method; (4) torso angle up and leg swing right method; (5) torso angle up and leg swing left method; (6) torso angle up and upper body roll right method; and (7) torso angle up and upper body roll left method. A given patient may utilize one or more of the foregoing methods or a variation thereof, but typically one will dominate. Other support exiting behaviors are possible and can be accounted for where relevant.

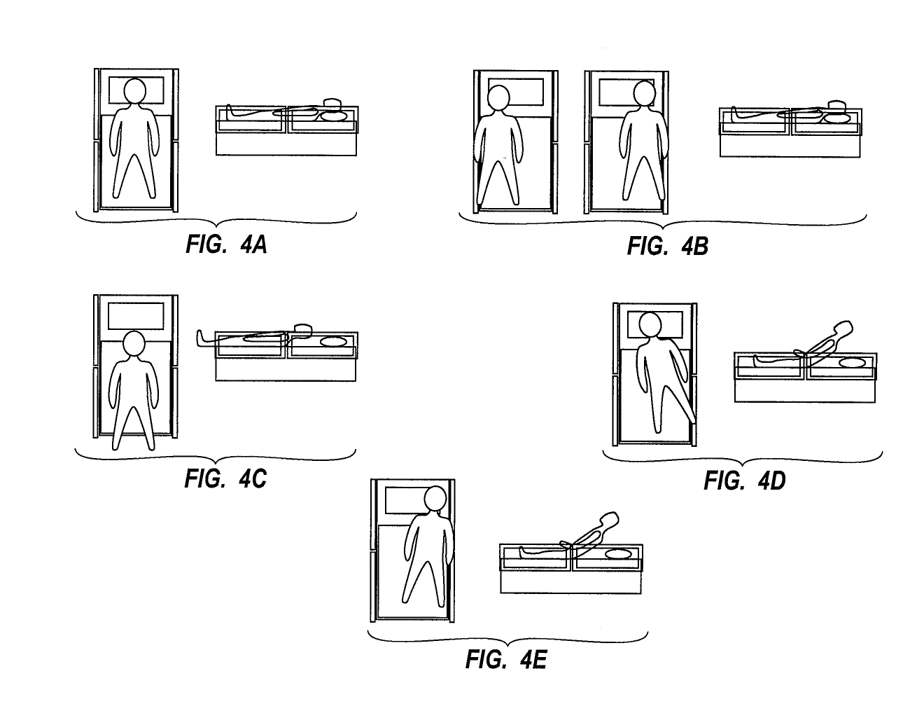

FIG. 4A schematically illustrates a normal resting position of a patient lying flat on a bed. FIGS. 4B-4E schematically illustrate positions associated with various bed exiting positions, movements or behaviors. FIG. 4B roughly depicts the position of a patient that has engaged in the bed slide method of bed exiting. A notable feature is the distance between the patient's head and the pillow or headboard. FIG. 4C illustrates left and right side rail roll methods in which the patient's body moves to the side or left side rail preparatory to bed exiting. FIG. 4D illustrates the torso up and leg swing left method of bed exiting, which is characterized by upward movement of the torso coupled with movement of the left leg toward the edge of the bed. The torso up and right leg swing method is simply the mirror image of that shown in FIG. 4D. FIG. 4E illustrates the torso up and upper body roll left method, which is characterized by the patient's torso moving upward and the patient's body rolling to the left. The torso up and upper body roll right method would be the mirror image of that shown in FIG. 4E.

Each patient profile will contain one or more spatial parameters associated with the one or more support exiting behaviors that are known for each patient. The spatial parameters relating to bed exiting may include data points pertaining to one or more of the seven common bed exiting behaviors noted above. Image parameters relating to exiting of other supports can be tailored to behaviors that are typical for patients exiting such supports. Patient profiles may include idiosyncratic information that is specific to a particular individual (e.g., base on patient height, weight, speed of movement, length of limbs, number of operable limbs, and/or personal habits of position and/or movement while support exiting).

By way of example, as illustrated a spatial parameter that corresponds to the bed slide method of bed exiting is the distance from a head feature to the top of the bed (e.g., headboard) (see FIG. 4B). Spatial parameters corresponding to the side rail roll methods (left or right) for bed exiting include: (a) the torso positioned primarily to the right or left of the bed and (b) the hand and/or arm on or over (i.e., covering or blocking the view of) the left or right bed rail for a given period of time (see FIG. 4C). Spatial parameters corresponding to the torso up and leg swing methods (left or right) of bed exiting include: (a) the head elevated from a flat position and (b) right or left legs and/or feet breaking a vertical bed edge plane (see FIG. 4D). Spatial parameters corresponding to the torso up and upper body roll methods (left or right) of bed exiting include: (a) the head elevated from a flat position; (b) torso positioned primarily to the right or left portion of the bed; and one or both of (c₁) the left or right hand and/or arm on or over (i.e., covering or blocking the view of) the left or right bed rail for a given period of time and/or (c₂) the head breaking a vertical plane of the left or right side rail (see FIG. 4E). In addition to patient body position, time of duration of a limb or body part at a specified location relative to a critical region of the support may also play a roll in determining bed or other support exiting.

A second step 302 of method 300 involves associating a corresponding patient profile with the particular patient being monitored. The use of RFID, ultrasound, or other patient identification and tracking devices may assist in identifying which patient profile corresponds to the patient being monitored. For example, if a patient moves from room to room over time, different monitoring equipment in the various rooms can all monitor the same patient at different times, while comparing patient position and/or movements with specific profile data for that patient, because the patient is associated with a patient identification and tracking device that emits a uniquely encoded signal. Such association may alternatively be made (e.g., entered manually into a computer) by hospital staff whenever a patient occupies a particular room.

A third step 303 of method 300 involves continuously monitoring a patient resting on a support by capturing a series of images of the patient and surroundings and sending a data stream (e.g., video feed) to a computer system for analysis. Since both motion video recording devices and still photo devices are capable of taking individual frames, the distinction between the two is simply in the speed with which individual frames are taken (i.e., the time interval between frames). Thus, both motion video recording devices and still photo devices can be used to send a continuous data stream to the analyzing computer system.

A fourth step 304 of method 300 involves analyzing the data stream (e.g., frames of video data) to determine patient position and/or movement and comparing them to patient profile data relating to the support exiting behavior of that patient. As discussed above, such computer-implemented analysis of position and/or movement may be carried out using a grid monitoring system (GMS), which compares the relative position of one or more body parts in relation to stationary background objects, such as critical or predefined support zones. The use of patient specific profiles enables the computer system to more accurately detect and distinguish between behaviors that are indicative or predictive of patient support exiting and those which are not as compared to methods that are not patient specific but utilize the same sets of analytical limits for all patients. In this way, the incidences of false positives and false negatives are significantly reduced or substantially eliminated.

In the event that behavior consistent with predicted support exiting is detected, a fifth step 305 of method 300 is triggered. This step includes initiating an appropriate response in an attempt to prevent or mitigate harm to the patient. Exemplary responses include sending an alarm and/or video feed to a nurse's station, establishing one- or two-way communication between the patient, sending a pre-recorded message to the patient, sending notification to a nearby caregiver who can provide direct physical intervention, sounding an alarm, and the like. It may even be appropriate in some cases to activate an automated restraint device that is able to keep the patient from exiting the support until a caregiver is able to arrive and provide assistance.

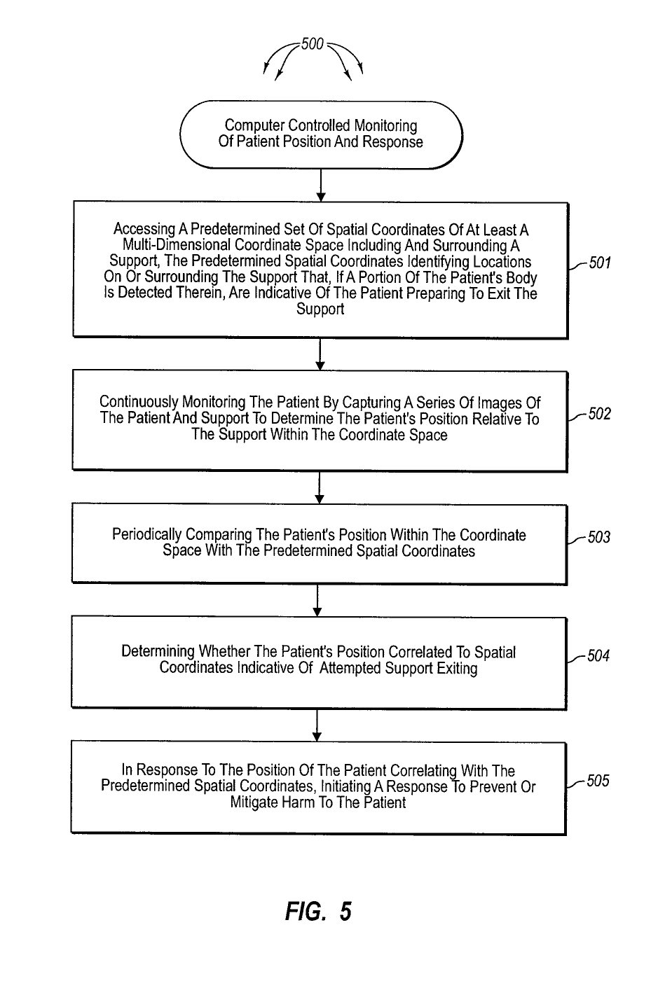

FIG. 5 is a flow chart that schematically illustrates an exemplary computer-controlled method 500 for patient monitoring and response relative to exiting a support. Method 500 includes a first act 501 of providing or accessing a predetermined set of spatial coordinates of at least a two- or three-dimensional coordinate space including and surrounding a support that are predictive of a patient preparing to exit the support. A two-dimensional coordinate space can be analyzed when a single camera is available. A three-dimensional coordinate space can be analyzed when two or more cameras are available.

High risk motion clients and/or cameras can be arranged at different viewing angles relative to a support to provide stereoscopic image. An arrangement with different angles can yield superior real time monitoring of patient location (e.g., in a three dimensional Cartesian coordinate space, such as, X, Y and Z coordinates). In some embodiments, a camera faces a patient to measure side to side and up and down movement. The first camera can also provide video conferencing capability if combined with a TV monitor. A second camera is on the side of patient, (possibly facing the door) to measure longitudinal and up and down movement (and optionally to film persons entering room). An aerial camera can detect longitudinal and side-to-side movements.

The predetermined spatial coordinates identify locations on or surrounding the support that, if a portion of the patient's body is detected in the locations (e.g., for a prescribed time duration), are indicative or predictive of the patient preparing to exit the support. The predetermined set of spatial coordinates may be accessed or provided as part of a patient specific profile accessed by a computer system performing the support exiting analysis. The computer system may be the facility master computer or it may be an in room controller client associated with the patient's room.

Method 500 further includes an act 502 of continuously monitoring the patient occupying the support by sending a series of video images to a computer system for determining the position of the patient at a given moment. For example, high risk motion clients (which may include cameras) as discussed above can continuously take and send a video data stream relating to patient location and/or movement relative to a bed, wheelchair, chair, gurney, recliner, other furniture, or other support to a computer system. The computer system can temporarily store (e.g., temporarily buffer) the series of images and then process the images to determine the position and/or movement of the patient at a given moment.

Method 500 includes an act 503 of periodically comparing the position of the patient with the predetermined set of spatial coordinates (e.g., continuously or at intervals sufficiently close together as to be sufficiently precise or vigilant relative to preventing harm to a given patient). The time duration of the intervals may depend on the speed with which a given patient is known to exit a support. According to one embodiment, one or more video data streams are buffered within the computer system for a time sufficient to allow for detailed analysis of the patients location and/or movements. The buffered video data streams are typically deleted without being permanently recorded or archived to preserve patient privacy.

Act 504 involves determining whether or not the patient's position and/or movements are consistent with the predetermined set of special coordinates that, if invaded by the patient, are predictive of support exiting. The analyzing computer system can utilize various image analysis and processing algorithms to translate portions of buffered images into the predetermined set of spatial coordinates.

In response to the patient's position and/or movements correlating with the predetermined set of special coordinates predictive of support exiting, act 505 includes initiating a response to prevent or mitigate harm to the patient as described herein. The computer system may control automated alerts, warnings, instructions, one- or two-way communications, prerecorded messages, verifying whether an appropriate intervention was actually completed, and any other aspects of the response as desired. According to one embodiment, an in room controller client can override any current programming on an A/V interface client (e.g., a television program) with a message for the patient to cease current movements consistent with support exiting.

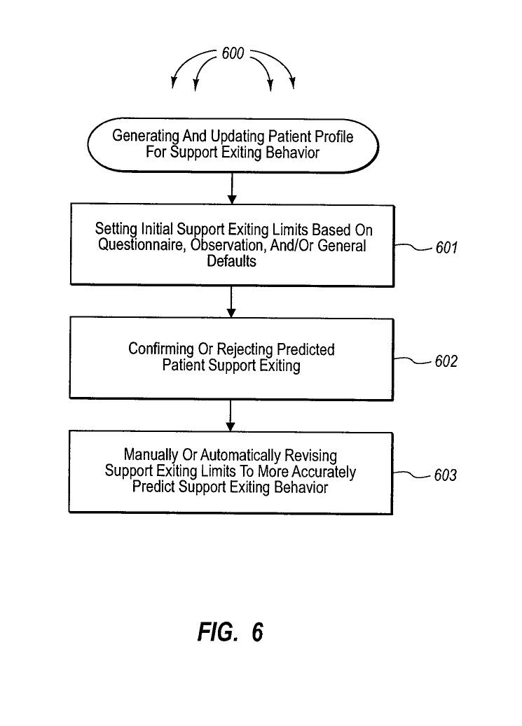

FIG. 6 is a flow chart that schematically illustrates an exemplary method or sub-routine 600 of generating and updating a patient profile for support exiting behavior. A first act or step 601 involves setting initial support exiting limits based on information learned from or about the patient (e.g., as a result of a patient or relative completed questionnaire, observation by a qualified provider, general defaults, and the like). It is understand that the initial limits are advantageously modified as more information is gathered over time regarding a patient's actual support exiting habits while at one or more facilities.

Accordingly, a second act or step 602 includes actually monitoring a patient while resting on a support as discussed above and then either confirming or rejecting an alert of predicted patient support exiting. From one or more confirmations or rejections of predicted bed exiting, additional information regarding the specific support exiting habits of the patient can be learned.

A third act or step 603 includes manually or automatically revising or updating previously set support exiting limits in order to more accurately predict support exiting behavior by patient in question. In some cases, the computer system may appropriately alter patient profile data relating to bed exiting so long as it does not substantially increase the risk of unassisted support exiting. In other cases, patient profile data relating to bed exiting may be altered manually by a qualified individual or committee who analyzes data generated during predicted support exiting events. Limits can be established initially, or pre-existing limits may be tightened or loosened, in response to incidences of false positives and/or false negatives relative to support exiting.

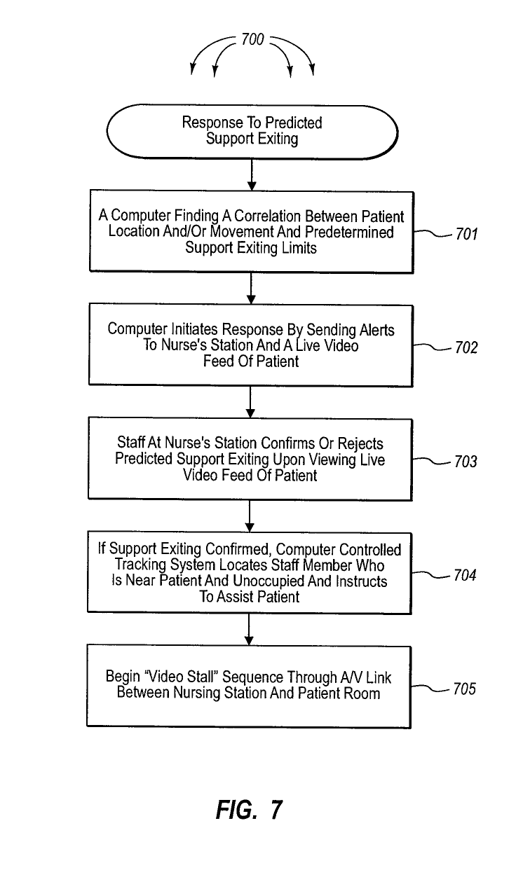

FIG. 7 is a flow chart that schematically illustrates an exemplary method or sub-routine 700 of a response to predicted patient support exiting. In a first act or step 701, a computer system finds a correlation between a patient's location and/or movements and predetermined limits for that patient contained in a patient specific profile. A second act or step 702 involves a computer initiating a response by sending an alert to both the patient's room (to warn of a breech in privacy) and a nurse's station along with a live (i.e., real time) video feed of the patient's room to the nurse's station. In a third act or step 703, a staff member at the nurse's station confirms or rejects the predicted support exiting upon viewing the live video feed of the patient's room. In a fourth act or step 704, if support exiting is confirmed, a computer-controlled tracking system locates an unoccupied staff member who is assigned and/or near the patient's room and instructs the staff member to assist the patient.

FIG. 8 is a flow chart that schematically illustrates an exemplary monitoring and response decision chart relative to bed exiting. The patient is continuously or periodically monitored, and the patient's position and/or movements are analyzed. As long as positions and/or movements predictive of bed exiting are not detected, monitoring continues. Of course, monitoring may also continue even after bed exiting is predicted in order to send a live video feed to a central station and/or determine an escalation of events.

If predicted patient bed existing is detected by the analyzing computer system, an alert is sent to a nurse's station as well as a live video feed of the patient for verification of actual bed exiting. Prior to or at the same time, an alert is sent to the patient's room of potential third party viewing of the patient (e.g., to protect patient privacy). If no verification (i.e., confirmation or denial) is sent to the computer system within a predetermined time period, an automated response is initiated. If verification is sent, the computer determines whether bed exiting is confirmed or denied. If bed exiting is denied, the computer system resumes normal patient monitoring. If bed exiting is confirmed, further intervention is initiated.

The escalation of intervention to assist a patient who is in the process of bed exiting may include establishing one- or two-way communication between the confirming staff member and the patient. It may also include sending an alert to a nearby or assigned staff member for direct physical intervention. An RFID, ultrasound, or other staff tracking device can be used to verify that physical intervention was carried out as prescribed. The assisting caregiver may press a confirm button on a patient care interface device connected to the computer system, or the caregiver may provide oral confirmation to the staff member at the nurse's station. The staff member at the nurse's station may view the live video feed from the patient's room to confirm successful intervention. If intervention is confirmed, the response is complete. If intervention is not confirmed, the response may include sending one or more additional alerts to other nearby staff members for direct physical intervention.

III. General Systems and Methods for Patient, Personnel, Visitor and Asset Monitoring and Care

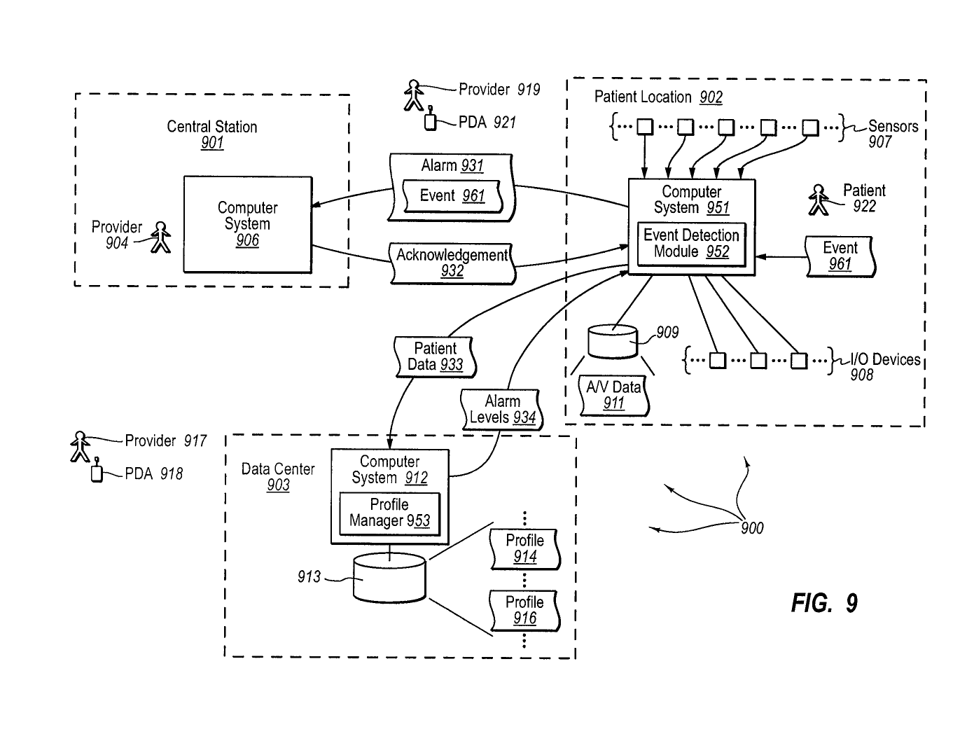

The inventive systems and methods for monitoring and responding to patient support exiting may form part of a more comprehensive patient monitoring and wellness system. FIG. 9 illustrates an exemplary computer architecture 900 that facilitates monitoring and event response management at a healthcare facility. Computer architecture 900 includes computer systems 906, 912, and 951. Each computer system can be connected to a network, such as, for example, a Local Area Network (“LAN”), a Wide Area Network (“WAN”), or even the Internet. Thus, the various components can receive data from and send data to each other, as well as other components connected to the network. Networked computer systems may themselves constitute a “computer system” for purposes of this disclosure.

Networks facilitating communication between computer systems and other electronic devices can utilize any of a wide range of (potentially interoperating) protocols including, but not limited to, the IEEE 802 suite of wireless protocols, Radio Frequency Identification (“RFID”) protocols, ultrasound protocols, infrared protocols, cellular protocols, one-way and two-way wireless paging protocols, Global Positioning System (“GPS”) protocols, wired and wireless broadband protocols, ultra-wideband “mesh” protocols, etc. Accordingly, computer systems and other devices can create message related data and exchange message related data (e.g., Internet Protocol (“IP”) datagrams and other higher layer protocols that utilize IP datagrams, such as, Transmission Control Protocol (“TCP”), Remote Desktop Protocol (“RDP”), Hypertext Transfer Protocol (“HTTP”), Simple Mail Transfer Protocol (“SMTP”), etc.) over the network.

In some embodiments, a multi-platform, multi-network, multi-protocol, wireless and wired network architecture is utilized to monitor patient, staff, visitor, and asset locations and movements within a facility. Computer systems and electronic devices may be configured to utilize protocols that are appropriate based on corresponding computer system and electronic device on functionality. For example, an electronic device that is to send small amounts of data a short distance within a patient's room can be configured to use Infrared protocols. On the other hand, a computer system configured to transmit and receive large database records can be configured to use an 802.11 protocol. Components within the architecture can be configured to convert between various protocols to facilitate compatible communication. Computer systems and electronic devices may be configured with multiple protocols and use different protocols to implement different functionality. For example, an in room controller can receive patient data via infrared from a biometric monitor and then forward the patient data via fast Ethernet to a data center for processing.

In some environments, ultrasound technologies, such as, for example, those developed by Sonitor Technologies, may be preferred for monitoring patient, staff, and asset locations, movements, and interactions within a facility. Ultrasound waves can be blocked by normal walls, are less likely to reflect off of metallic objects, and are less likely to interfere with sensate instruments. For example, ultrasound waves can be confined to a room (e.g., a patient room) where they originate. When using ultrasound receivers and detectors, various Digital Signal Processing (DSP) algorithms can be used to convert ultrasound waves into meaningful digital data (e.g., for transport on a wired network). The DSP algorithms can be configured to ensure that ultrasound detectors interpret ultrasound waves without risk of interference from any environmental noise or other signals nor interference with sensitive instruments.

However, in other environments the increased range of RFID may be preferred for monitoring patient, staff, and asset locations, movements, and interactions within a facility. For example, since RFID signals can pass through walls, RFID detection systems can be implemented with fewer detectors.

Computer system 906 can be physically located at a central station 901 of a healthcare facility, which is, for example, a nurse's station. Provider 904 (a nurse or other healthcare worker) can be physically located near computer system 906 such that provider 904 can access electronic messages (e.g., alarms) presented at computer system 906. Other healthcare providers, for example, providers 917 and 919, can be physically located in other parts of a healthcare facility. Healthcare providers can move between different locations (e.g., central station 901, patient rooms, hallways, outside the building, etc.). Accordingly, healthcare providers can also carry mobile computer systems (e.g., laptop computers or PDAs 918 and 921) and other types of mobile devices, (e.g., pagers, mobile phones, GPS devices, ultrasound, or RFID devices). Thus, as healthcare providers move about a healthcare facility they can still access electronic messages (e.g., alarms) and send messages.

Computer system 951, storage device 909, sensors 907, and I/O devices 908 can be physically located at patient location 902, such as, for example, patient rooms, hallways, and other appropriate locations throughout or outside a healthcare facility. For example, patient location 902 can be a room of a patient 922. Sensors 907 can include various types of sensors, such as, for example, video cameras, still cameras, microphones, pressure sensors, acoustic sensors, temperature sensors, heart rate monitors, conductivity sensors, RFID detectors, ultrasound detectors, global positioning sensors (“GPS”), manual assistance switches/buttons, bed sensors, handrail sensors, mattress sensors, location sensors, oxygen tank sensors, etc. Thus, although depicted separately, I/O devices 908 can also be sensors.

Some sensors 907 can be stationary (e.g., mounted at patient location 902) such that the sensors sense patient (or provider) characteristics when a patient (or provider) is within a specified vicinity of the sensor 907. For example, characteristics of a patient's gait can be observed when the patient walks by a video camera or other sensor. A patient's gait can also be monitored by measuring the time it takes a patient to move between zones. Other sensors can be mobile and move with a patient, provider, asset or visitor they move about a healthcare facility. For example, a heart rate monitor can be attached to a patient and move with the patient to continuously monitor the patient's heart rate. As a patient, provider or asset move about a healthcare facility, different combinations of stationary and mobile sensors can monitor the patient, provider or asset at different locations and/or times.

Each of sensors 907 can provide input to computer system 951. Event detection module 952 can monitor inputs from sensors 907 and process inputs from sensors 907 to detect if a combination of inputs indicates the occurrence of a potentially actionable event. Detecting the occurrence of an event can trigger the transfer of various electronic messages from computer system 951 to other networked computer systems. For example, electronic messages (alarm messages) can be transferred to other computer systems to alert health care providers of an actionable event. Alternatively or in addition, electronic messages including patient data can also be transferred to other computer systems that process the patient data (e.g., computer systems that maintain patient risk profiles).

Combinations of different types and/or numbers of sensors 907 can be used to detect patient conditions, such as, for example, bed exiting, changes in gait, social interaction, breathing, etc. For example, RFID zones or ultrasound zones separated by specified distances can be used to monitor speed or interruptions in speed as a patient walks. Image analysis can determine the manner of a patient's walking.

Computer system 951 can buffer sensor input at storage device 909 for some amount of time before discarding the input.

Additionally, in response to detecting the occurrence of an event, computer system 951 can (in addition to monitoring and processing sensor input) archive sensor input. For example, computer system 951 can selectively actuate the archival of audio/video (“AV”) data from I/O devices 908 at storage device 909 based on a combination of inputs at patient location 902 (e.g., indicative of an actionable event). Buffered and/or archived sensor input can provide the basis for patient data that is subsequently transferred to other computer systems.

Event occurrences can be detected in accordance with a risk profile associated with a monitored patient. Thus, a combination of inputs detected as the occurrence of an (actionable) event for one patient is not necessarily detected as the occurrence of an (actionable) event for another patient, and vice versa. An actionable event can be detected when a specified alarm level for a given patient is satisfied. For example, a specified combination of risk indicating vital signs can cause an actionable event to be detected.

Computer system 912 and storage device 913 can be physically located at data center 903. Storage device 913 can store patient risk profiles (e.g., profiles 914 and 916) for patients. Profile manager 953 can receive patient data sent to computer system 912 (e.g., in response to a detected event) and refine a corresponding patient risk profile in accordance with the patient data. Accordingly, as data related to a patient changes, the patient's risk profile can be modified to indicate changed risks for the patient. Risk profiles for a patient can be iteratively (and automatically) refined as patient data for the patient is received. Algorithms for refining profiles can be recursed on a per iteration basis.

Patients, providers, visitors and assets may carry RFID or ultrasound transmitting devices, each having a unique signature such that an RFID or ultrasound transmitting device can be used to determine the location of a patient, provider, visitor or asset within a healthcare facility. RFID and ultrasound transmitting devices can be non-removable, such as, for example, a bracelet or medical ID badge or removable, such as, for example, an employee badge.

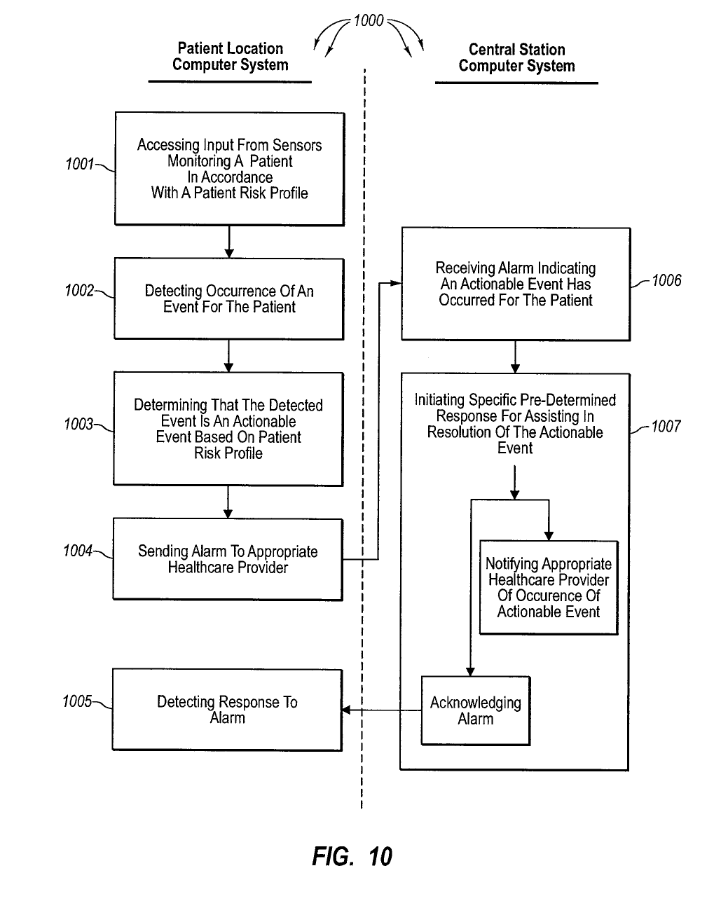

In some embodiments, assistance is at least partially provided through communication among and between computer systems. FIG. 10 illustrates a flow chart of a method 1000 for managing a response to an actionable event in a healthcare facility. Method 1000 will be described with respect to the components and data in computer architecture 900.

Method 1000 includes an act 1001 of accessing input from sensors monitoring a healthcare facility patient in accordance with a patient risk profile. For example, computer system 951 can access input from sensors 907 that monitor patient 922 for one or more conditions that, when combined, indicate occurrence of an actionable event in accordance with profile 914. An actionable event can be a detected medical condition, patient location, patient movement, support exiting behavior, healthcare provider location, etc. For example, a series of movements in a three dimensional coordinate space can indicate that a patient is attempting to exit support.

Profile 914 can be a recursively refined profile indicating actionable (and non-actionable) events for patient 922 in accordance with previously collected data relating to patient 922. Alternatively, when no patient data has previously been collected for patient 912 at a particular facility, profile 914 can indicate actionable (and non-actionable) events based on historical patient data.

Method 1000 includes an act 1002 of detecting occurrence of a patient related event for the patient. For example, event detection module 951 can detect the occurrence of event 961 for patient 922 (from the combined input of sensors 907). Method 1000 includes an act 1003 of determining that the detected event is an actionable event based on the patient risk profile. For example, event detection module 952 can determine that event 961 is actionable based on profile 914. Profile manager 953 can create alarm levels 934 and send alarm levels 934 to event detection module 952. Alarm levels 934 can include one or more combinations of values for inputs 907 that indicate an actionable event based on profile 914. When one or more monitored values satisfy an alarm level, an actionable event is detected.

Method 1000 includes an act 1004 of sending an alarm to an appropriate healthcare provider. For example, computer system 951 can send an alarm 931, including event 961, to computer system 906 to indicate the occurrence of event 961 to healthcare provider 904. Method 1000 also includes an act 1006 of receiving an alarm indicating an actionable event has occurred for the patient. For example, computer system 906 can receive alarm 931 indicating that event 961 (an actionable event) has occurred for patient 922. Thus, computer system 906 is notified that an alarm was generated in response to input from sensors 907 monitoring the occurrence of event 961 for patient 922 in accordance with profile 914.

Method 1000 includes an act 1007 of initiating a specific pre-determined response for assisting in resolution of the actionable event. For example, computer system 906 can initiate a specific pre-determined response for assisting in resolution of event 961 in response to receiving alarm 931. A response can include notifying an appropriate health care provider of the occurrence of the actionable event. For example, in response to receiving alarm 931, computer system 906 can present an audio and/or video indication of event 961 at central station 901, such as, for example a video display and speakers. Alternately, or in addition, one or more of PDAs 918 and 921 can also receive alarm 931 and present an audio and/or video indication of event 961 to providers 917 and 919 respectively.

A response can also include acknowledging the alarm. For example, computer system 906 can send acknowledgment 932 to computer system 951. An acknowledgment can include commands for opening one or two-way communication between a healthcare provider and patient location 902 (e.g., using I/O devices 908). For example, provider 904 can input commands at computer system 906 to open communication from central station 906 to patient location 902. Similarly, providers 917 and 919 can input commands at PDA's 918 and 921, respectively, to open communication from their locations to patient location 902. Communication can be used to send instructions to a patient, ascertain whether a patient is coherent, responsive to commands or instructions, etc.

A response can also include a provider responding to the location of a patient. For example, in response to detecting that patient 922 has fallen, might fall, or otherwise requires assistance (e.g., by a patient controlled call device), provider 904 can respond to patient location 902. RFID and/or ultrasound detectors at patient location 902 can detect an RFID transmitting device and/or ultrasound transmitting device corresponding to provider 904 to verify response by provider 904 to a patient need (e.g., comprising act 1005 of method 1000).

Expiration of a time interval can trigger some actionable events. For example, movement of bed bound patients or administration of medicine can be required at specified intervals. Computer system 951 can send an alert to computer system 906 (or other appropriate computer systems) when a time interval expires or is about to expire.

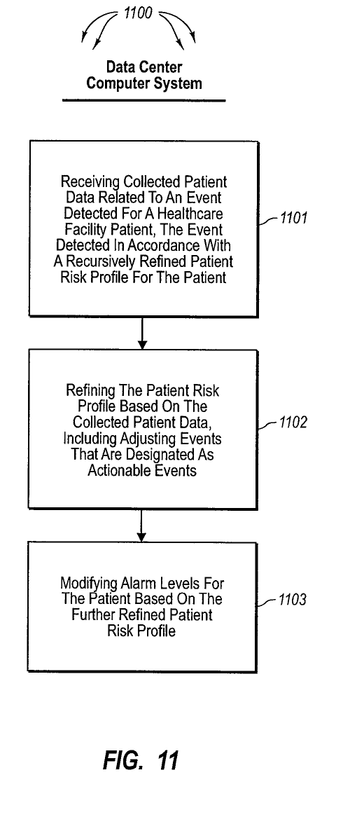

In some embodiments, stored profiles are risk profiles that include recursively refined patient alarms levels indicative of actionable events requiring a response. FIG. 11 is a flow chart that illustrates a method 1100 for maintaining patient risk profiles and associated alarms levels for a patient at a healthcare facility. Method 1100 will be described with respect to the components and data in computer architecture 900.

Method 1100 includes an act 1101 of receiving collected patient data related to a detected event for a healthcare facility patient. The event is detected in accordance with a recursively refined patient risk profile for the patient based on previously collected patient data. For example, computer system 912 can receive patient data 933 related to event 961 for patient 922. As previously described, event 961 can be detected in accordance with profile 914 based on previously collected patient data for patient 922 (or on historical default data). Collected patient data is collected from a plurality of sensors 907 monitoring the patient for various conditions that, when combined or considered individually, indicate occurrence of an event 961. Although event 961 may be an actionable event, embodiments of the invention can also receive data in response to non-actionable events 961. For example, some events may trigger refinement of a patient risk profile without triggering an alarm.

Method 1100 includes an act 1102 of refining the patient risk profile further based on the collected patient data. Further refinement includes adjusting events that are designated as actionable events. For example, profile manager 953 can refine patient risk profile 914 based on patient data 933. Profile manager 953 can adjust events that are designated as actionable events for patient 922. Profile manager 953 can iteratively refine profile 914 through recursive application of profile refinement algorithms.

Method 1100 includes an act 1103 of modifying alarm levels for the patient based on the further refined patient risk profile such that an appropriate health care response can be provided for alarms indicative of actionable events. For example, profile manager 953 can adjust alarm levels 934 for patient 922 based on refinements resulting from patient data 933. Alarm levels 934 can cause an appropriate healthcare provider to be notified when actionable events related to patient 922 occur. Alarm levels 934 can differ from previous alarm levels for patient 922 as a result of refinements to profile 914 to adjust risk based on patient profile 933. In some embodiments, a feedback loop continually updates patient profiles to fine tune the monitoring of patient conditions. For example, monitoring for bed exiting can begin with common preset values that are updated over time to create unique or verified information for each patient.

A decision algorithm can be used to adjust parameter values that will cause an actionable event. If an actionable event is appropriately detected (a positive), parameters can be made more restrictive such that the standard is lowered for detecting the actionable event in the future. For example, if a patient has fallen when exiting a bed, the values for detecting a bed exit can be made more restrictive. On the other hand, if an actionable event is inappropriately detected (a false positive), parameters can be made less restrictive such that the standard is raised for causing or detecting the actionable event in the future. When no actionable event is detected (a negative) for some time period, the parameters can also be made less restrictive such that the standard is raised for causing or detecting the actionable event in the future.

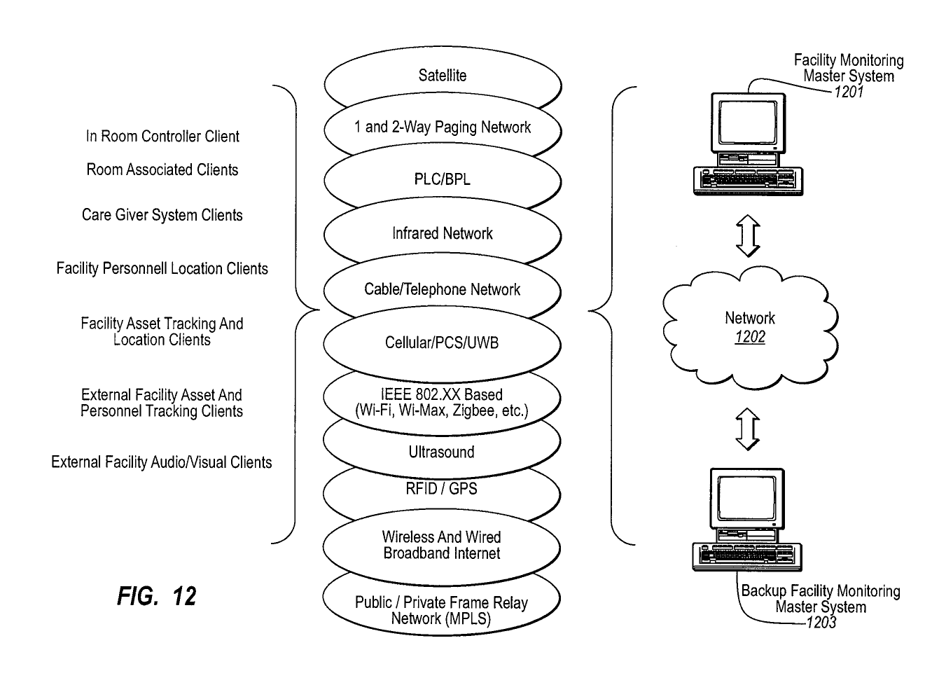

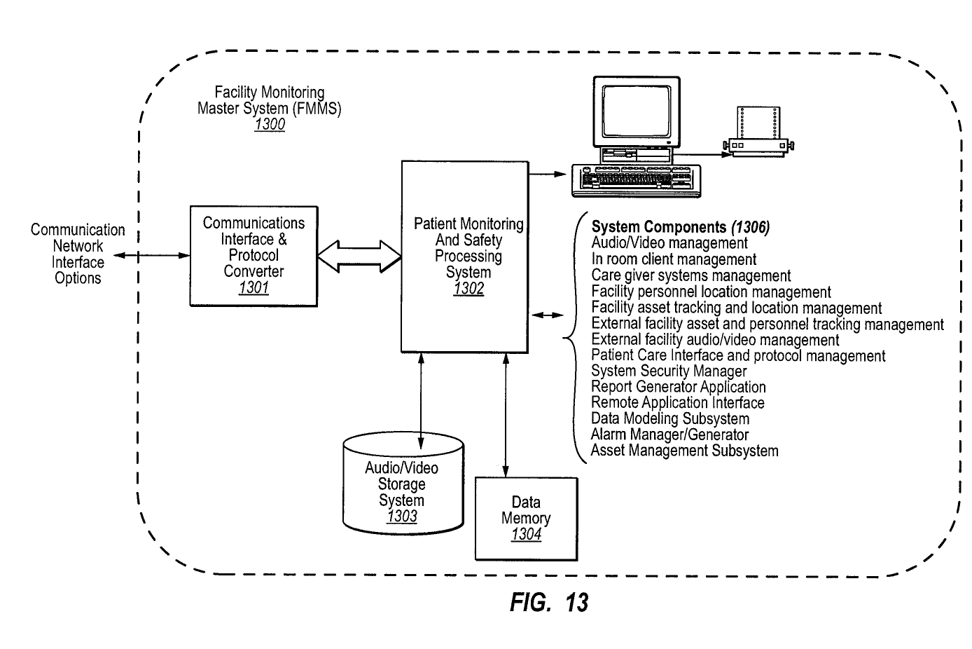

FIG. 12 illustrates an example of various network protocols that can be used to facilitate communication between computer systems in a healthcare facility. The various depicted protocols can be used to facilitate communication via network 1202 for various clients to facility monitoring master system 1201 and to a backup facility monitoring master system 1203. The various depicted protocols can also be used to facilitate communication between components in computer architecture 900 (FIG. 9) and in patient room 200 (FIGS. 2A-2C).

FIG. 13 illustrates an example of a facility monitoring master system 1300. Facility monitoring master system 1300 can be included in a data center, such as, for example, data center 903 (FIG. 9). Communications interface and protocol converter 1301 can receive communication in accordance with one of the various protocols of FIG. 12 and can convert the communication for compatibility with patient monitoring and safety processing system 1302. Audio/video storage system 1303 can store archived A/V data (e.g., archived in response to detection of an actionable event). System components 1306 lists example components that can be included in patient monitoring and safety processing system 1302 to facilitate the principles of the present invention. Accordingly, embodiments of the present invention provide an automated architecture for health care providers to more effectively provide care and wellness to patients.

IV. Optional Enhancements

A. Maintaining Stored Profiles

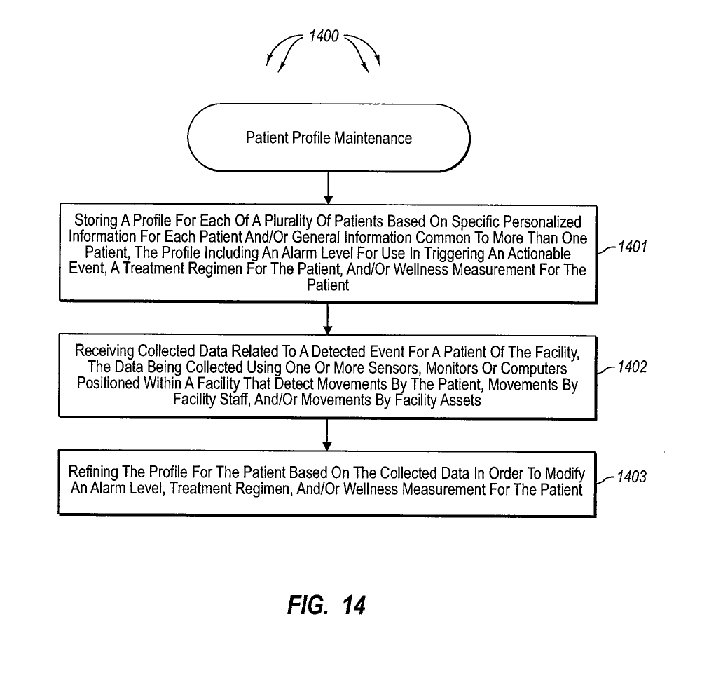

FIG. 14 illustrates a flow chart of a method 1400 for maintaining stored profiles for patients of a healthcare facility. Method 1400 will be described with respect to the components and data in computer architecture 900 (FIG. 9) and a hypothetical patient room (e.g., room 200 of FIGS. 2A-2C).

Method 1400 includes an act 1401 of storing an initial profile for each of a plurality of patients at a facility based on at least one of specific personalized information for each patient or general information common to more than one patient, the profile including at least one of an alarm level for use in triggering an actionable event, a treatment regimen for the patient, or wellness measurement for the patient. For example, storage device 913 can store profiles 914, 916, etc. for patients of a hospital or other healthcare facility. The profiles 914, 916, etc. can include alarm levels for the patients for use in triggering actionable events (e.g., based on the patient's condition, behavior, persons in the patient's vicinity, etc.), patient care, patient wellness, or a patient measurement (e.g., blood pressure, temperature, etc.)

Method 1400 includes an act 1402 of receiving collected data related to a detected event for a patient of the facility, the data being collected using one or more sensors, monitors or computers positioned within the facility that detect at least one of movements by the patient, movements by facility staff, or movements by facility assets. For example, computer system 912 can receive collected data (e.g., patient data 933) from computer system 95 1. Computer system 951 can collect data for a patient and/or related to a detected event for a patient event from one or more of sensors 907, I/O devices 908, etc. For example, sensing devices in a patient room can collect patient data for a patient. An in room controller client can appropriately aggregate and format data for transmission to data center 903.

Method 1400 includes an act 1403 of refining the profile for the patient based on the collected data in order to modify at least one of an alarm level, a treatment regimen, or a wellness measurement for the patient. For example, profile manager 953 can refine profile 914 based on patient data 933 to modify at least one of an alarm level, a treatment regimen, or a wellness measurement for a patient.

In some embodiments, stored profiles are risk profiles that include recursively refined patient alarms levels indicative of actionable events requiring a response. FIG. 11, discussed above, illustrates a flow chart of a method 1100 for maintaining alarms levels in a patient risk profile for a patient of a healthcare facility.

B. Providing Patient Assistance

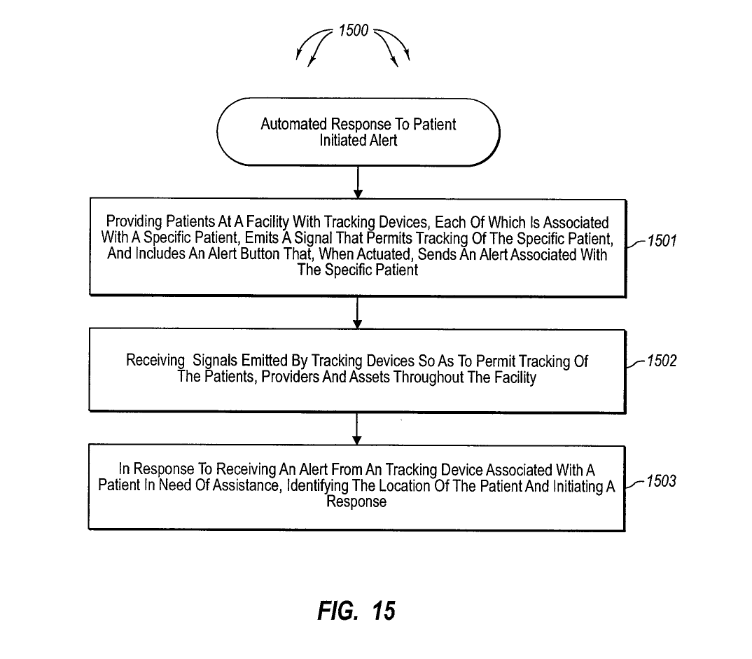

FIG. 15 a flow chart which illustrates an exemplary method 1500 for providing assistance for a patient in need thereof. Method 1500 will be described with respect to the components and data in computer architecture 900 (FIG. 9). Method 1500 includes an act 1501 of providing the patients of a facility with tracking devices, each of which is associated with a specific patient, emits a signal that permits tracking of the specific patient, and includes an alert button that, when actuated, sends an alert associated with the specific patient. For example, as previously described, a patient staying in a room at the facility can be provided a tracking device (e.g., an RFID or ultrasound patient location bracelet) specifically assigned to the patient.

Method 1500 includes an act 1502 of receiving one or more signals emitted by one or more tracking devices so as to track the location of patients throughout the facility. For example, sensing devices 907 within the facility can receive an RFID signal, an ultrasound signal, an alarm signal, etc. from each patient tracking device. Each patient can be tracked and located in patient rooms and also throughout hallways, other common areas, and dangerous or otherwise restricted areas of a healthcare facility. Signals can be detected by RFID sensors, ultrasound sensors, etc., throughout a facility and relayed to computer systems that process the signals to generate appropriate electronic messages and notifications.

Method 1500 includes an act 1503 that includes, in response to receiving an alert from a tracking device associated with a patient in need of assistance, identifying the location of the patient and initiating a response. For example, in response to receiving an alert from a patient tracking device, the location of an assigned or nearby caregiver can be identified and appropriate physical intervention can be initiated. A computer system that processes the signal (e.g., an in room controller client) can generate an electronic message or notification that is sent to one or more other electronic devices corresponding to assigned or nearby healthcare providers (e.g., to computer system 906, PDA 918, PDA 921, etc.)

In response to using the alert feature, patient profiles can be updated to count the number of times each patient has initiated an authorized alert (e.g., an actual physical or medical emergency) versus an unauthorized alert (e.g., ordering room service). In order to provide for the specific needs of a patient, patient profiles data can be accessed and a predetermined prescribed response initiated (e.g., in the case of patients with special needs).

C. Selectively Archiving Patient Video Recordings

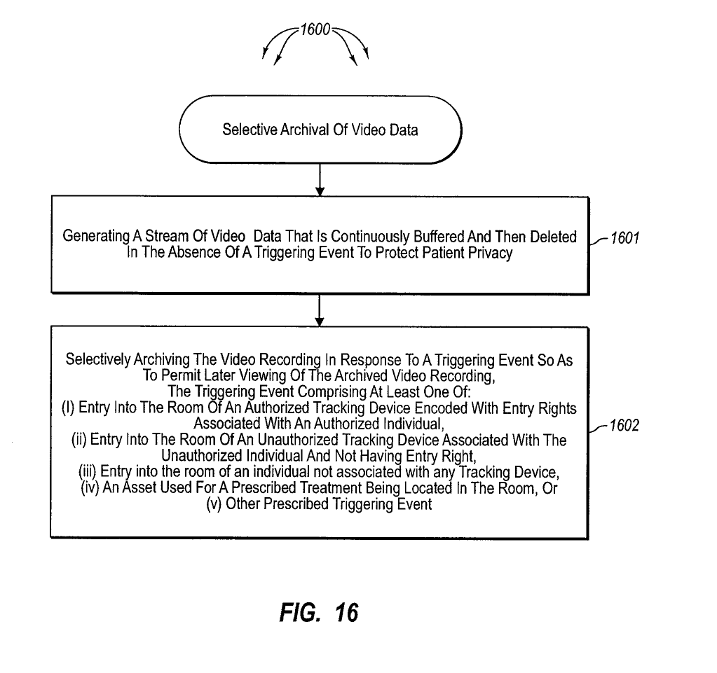

FIG. 16 is a flow chart that illustrates an exemplary method 1600 for selectively archiving a video recording of a patient in response to a triggering event. Method 1600 will be described with respect to the components in a typical patient room (e.g., room 200 of FIGS. 2A-2C).

Method 1600 includes an act 1601 of generating a video data stream of a patient's room at a healthcare facility, wherein the video data stream is continuously buffered and then deleted in the absence of a triggering event such that the buffered and deleted video recording is normally never viewed by an individual in order to protect patient privacy. For example, a computer system can use a circular buffer of a specified size such that after a prescribed amount of time (e.g., 3 to 5 minutes) older video data is overwritten by new video data within the buffer. By way of example, an in room controller and/or facility master can make a temporary or buffered video recording of received video data from one or more cameras positioned within a patient's room.

Method 1600 includes an act 1602 of selectively archiving the temporarily buffered video data stream in response to a triggering event so as to permit later viewing of the archived video recording. Video archival data can be stored at a healthcare facility or offsite. By way of example and not by limitation, the triggering event may comprise at least one of:
(i) entry into the room of an authorized tracking device encoded with entry rights associated with an authorized individual;
(ii) entry into the room of an unauthorized tracking device associated with an unauthorized individual not having entry rights;
(iii) entry into the room of an individual not associated with any tracking device;
(iv) an asset used for a prescribed treatment and associated with an tracking device being located in the room; and/or
(v) any other prescribed triggering event created by a given healthcare facility.

A tracking device can transmit any of a variety of different type signals, such as, for example, RFID signals or ultrasound signals.

D. Monitoring Patient Wellness

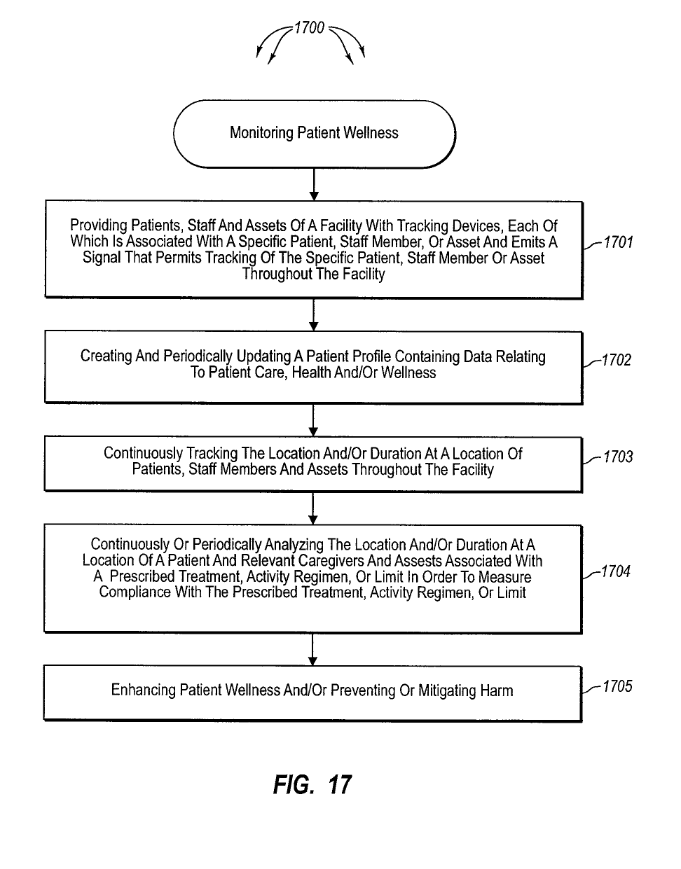

FIG. 17 illustrates a flow chart of a method 1700 for monitoring sufficiency of prescribed patient treatments, activities, or limits or other aspects of patient care, health and wellness. Method 1700 will be described with respect to the components and data in computer architecture 900.

Method 1700 includes an act 1701 of providing patients, staff, and assets of a facility with tracking devices, each of which is associated with a specific patient, staff member, or asset and emits a (e.g., RFID or ultrasound) signal that permits tracking of the assigned person or asset throughout the facility (e.g., location and also duration at a given location).

Act 1702 includes creating and periodically updating, for each of a plurality of patients, a profile containing data relating to patient care, health, and/or wellness (e.g., according to method 1400 of FIG. 14).

Method 1700 includes an act 1703 of continuously tracking the location of patients, staff, and assets throughout the facility. For example, a healthcare facility may include a plurality of RFID and/or ultrasound detectors located through out the healthcare facility. The location of the RFID and/or ultrasound detectors and assignment of RFID devices and/or ultrasound devices can be recorded and maintained in a computer system. As patients, staff members and assets move throughout the healthcare facility, the RFID and/or ultrasound detectors notify the computer system of RFID devices and/or ultrasound devices that are currently being detected. The computer system can correlate the location of each RFID device and ultrasound device, as well as the duration of each RFID device and ultrasound at a specific location, and determine whether prescribed health and wellness routines involving patients, staff, and assets have been properly carried out.

Method 1700 includes an act 1704 that includes, for a patient having at least one of a prescribed care, activity regimen, or limit, analyzing the location of the patient, assigned staff members, and relevant assets associated with the prescribed care, activity regimen, or limit in order to determine compliance with the prescribed care, activity regimen, or limit. Monitoring can be based on a facility blueprint, and prescribed locations of patients, staff and assets for a given task requirement. The location, movement and duration of providers and assets assigned to perform prescribed duties or tasks can be monitored to determine if prescribed duties or tasks are actually carried out and/or carried out properly (e.g., performed within predefined time guidelines or in proper location, such as bathing, assisted feeding, turning of bedridden patient to prevent bed sores, etc.).

According to act 1705, measures can be taken to enhance patient wellness and/or prevent or mitigate harm to a patient. For example, staff can be alerted to prevent or mitigate patient wandering into unauthorized or forbidden areas (e.g., other patient rooms, exiting facility, sensitive staff or equipment locations). Patient wellness events (e.g., face to face and/or video conferencing social interactions with visitors, other patients, staff; can determine the variety of such activities) can be chronicled and, if necessary, enhanced.

V. Exemplary System Logic

Solely by way of example only, the inventive systems and methods for patient monitoring and response may employ the following exemplary logic:

Assigned Limit Variables
[A]—head distance from headboard; initial value=30″
[B]—head elevation from flat/down position; initial value 12″
[C]—space between body and bedrail; initial value=5″—may need Small/Med/Large Values to reflect Patient body size
[D]—hand on bedrail time; initial value=5 seconds
[E]—bed bound/requires assist for exit; yes=1, no=0
[F]—patient room assignment for RFID/ultrasound
[G]—number of exit attempt for Torso Slide
[H]—number of exit attempt for Torso Up/Leg Sweep
[I]—number of exit attempt for Bedrail Roll
[J]—number of exit attempt for Unknown Method
[K]—family members video recorded; yes=1, no=0
[L]—other residents video recorded within room; yes=1, no=0
[M]—resident currently in facility; yes=1, no=0
[N]—requires movement assistance; yes=1, no=0
[O]—requires movement assistance every “X” hrs
[P]—does the resident require a special diet; yes=1, no=0
[Q]—does the resident require assistance during eating; yes=1, no=0
[R]—number of RFID/ultrasound presences in eating area during breakfast, lunch and dinner times per day
[S]—status of resident social interactions, maximum=10, minimum=0
[T]—requires device specific therapy every “Y” hrs
[U]—valid mobile emergency call button usage per month
[V]—unwarranted mobile emergency call button usage per month
[W]—is resident limited to movement within the facility; yes=1, no=0
[X]—is resident limited to movement within their room; yes=1, no=0

Image Analysis Outputs
(1) top of head to headboard distance (inches)
(2) head elevation from flat position (inches)
(3) leg in bed or out of bed (in/out)
(4) space between body and bedrail (inches)
(5) hand grasping bedrail duration (seconds)
(6) no body in bed (absent/present)

Alert Conditions for Bed Exiting
For all [E]=1
Torso Slide=(1)>[A]
Torso Up/Leg Sweep=(2)>[B] and (3)=out
Bedrail Roll=(4)<[C] and (5)>[D]
Bed Exit Has Occurred=(6) is absent and RFID/ultrasound [F] is positive

Action Taken for Positive Exit Alert

check RFID/ultrasound for Staff presence at Nursing Station
if no—then send pre-recorded message, alarm sent to closest shell staff RFID/ultrasound PDA, document, and go to Patient Profile Update
if yes—then request Alert Verification or Alert Rejection from staff
   if neither Verify or Reject is given within 30 seconds then send pre-recorded message, alarm sent to closest shell staff RFID/ultrasound PDA, document and go to Patient Profile Update
if yes and Alert Verification is Positive then
   alarm sent to closest shell staff RFID/ultrasound PDA
   Video/Audio link established with Nursing Station
   Patient Profile is Updated
if yes and Alert Rejection is Positive then
   Possible Patient Profile Update (loosen alert criteria)

Patient Profile Update for Bed Exiting
[A]=[A]−[G] until [A]=20″ then [A]=[A]
[B]=[B]−[H] until [B]=6″ then [B]=[B]
[C]=[C]+([I]/3) until [C]=9″ then [C]=[C]
[D]=[D]−([I]/3) until [D]=2 seconds then [D]=[D]

In-Room Camera Record On/Off Control
Camera Record is OFF until triggered by one of the following Actions:
   1. For [K]=yes and [L]=yes, the detection of an RFID/ultrasound that doesn't match with [F]
   2. For [K]=no and [L]=yes, the detection of an RFID/ultrasound that doesn't match with [F] and is not a Family RFID/ultrasound
   3. For [K]=yes and [L]=no, the detection of an RFID/ultrasound that doesn't match with [F] and is not a Resident RFID/ultrasound
   4. For [K]=no and [L]=no, the detection of an RFID/ultrasound that doesn't match with [F] and is not a Resident RFID/ultrasound or a Family RFID/ultrasound
   5. Alternatively for 3. and 4., a numeric coded “CAMMERA OFF” control pad could be accessible for each resident in each room then 3. and 4. would be deleted
   6. Door motion detector detects motion and no RFID/ultrasound is detected in the zone immediately positioned by the door—Alert Security
   7. Resident RFID/ultrasound [F] is detected but wide angle motion detector has not detected movement for over 12 hrs and [M]=1—Alert Nursing Station
An actively recording camera is STOPPED from further recording by one of the following Actions:
   1. The only detectable RFID/ultrasound signal is [F] and conditions (6) or (7) were not the source triggers
   2. No RFID/ultrasound detection and no detected movement in the room for >0.5 hrs
   3. Manual over-ride from Nursing Station

Bed Bound Movement Therapy
1. For [N]=1, NUM=number of Staff RFID/ultrasound visits to room per 24 hrs
2. For [N]=1, INTV=time period since last exiting of Staff RFID/ultrasound from room
3. If INTV>0.9*[O] then Alert Nursing Station
4. If INTV>1.3*[O] then Alarm Nursing Station and Document

Food/Nutrition Tracking
1. If [M]=1, [N]=0 and [R]=0 then Alarm and document
2. If [M]=1, [N]=0 and [R]=1 then Alert and RR=RR+1
3. If [M]=1, [N]=0 and RR>3 then Alarm and document

Resident Requires Assistance when Eating
1. If [M]=1 and [Q]=1 then For Count Staff RFID/ultrasound and Food Tray RFID/ultrasound in Patient Room during meal times QQ=QQ+1 per day, reset QQ=0 each night
2. If QQ<3 then Alert
3. If QQ<2 then Alarm and document
   (NOTE: if facility moves residents to another area for assistance with eating system will miss event)

Social Interaction Tracking
1. If RFID/ultrasound detected in Occupied Common Areas, then SI=SI+1
2. If Resident RFID/ultrasound detected in Room≠[F], then SI=SI+1
3. If detection of Assigned RFID/ultrasound in [F] and other Resident RFID/ultrasound in [F], then SI=SI+1
4. If detection of Family RFID/ultrasound in [F] while Assigned RFID/ultrasound is in [F], then SI=SI+1
5. At end of day, If SI>4 then [S]=[S]+1
6. At end of day, If SI=4 then [S]=[S]
7. At end of day, If SI=3 then [S]=[S]−1
8. At end of day, If SI=2 then [S]=[S]−2
9. At end of day, If SI=1 then [S]=[S]−3 and Alert Nursing Station
10. At end of day, If SI=0 then [S]=[S]−4 and Alarm Nursing Station and document
11. At end of day, If S<5 then Alert Nursing Station
12. At end of day, If S<1 then Alarm Nursing Station and Document

In-Room Therapy Requiring Special Equipment
1. Skip entire subroutine If [M]=0 or [T]=0
2. If Staff RFID/ultrasound and Device RFID/ultrasound are detected in [F], then TD=TD+1
3. If (current military time) >1.2*[T] and TD=0 then Alarm Nursing Station and document
4. If TD=1 then begin TDT=timer
5. If TDT>0.9*[T] then Alert Nursing Station
6. If TDT>1.2*[T] then Alarm Nursing Station and document
7. If TD=2 then TDT=0 and begin timer and TD=0
8. Loop back to 1.

RFID/Ultrasound Mobile Emergency Call Button
1. Unique Emergency RFID/ultrasound is detected
2. Individual Resident is Identified
3. Resident Location is Identified
4. Nursing Station is Alarmed
5. Location is compared to list of “wired” facility locations
6. If location is “wired”, video/audio link is established with Nursing Station
7. If RFID/ultrasound of Staff is present at Nursing Station and “wired” link existed, then wait 30 seconds for (VERIFY/REJECT) from Nursing Station before Alarm is transmitted to PDAs. Send Alarm immediately for VERIFY, no Alarm for REJECT.
8. If VERIFY then [U]=[U]+1, reset to [U]=0 at first of month
9. If REJECT then [V]=[V]+1, reset to [V]=0 at first of month
10. If [U]>1, document to staff “high risk resident”
11. If [V]>3, document to staff “resident requires counseling”

Resident Wandering Detection
1. If [W]=1 and solo Resident RFID/ultrasound (no associated Staff or Family RFID/ultrasound) is detected approaching or at exit then
   a. Alert Nursing Station
   b. Wait 30 seconds for VERIFY/REJECT
   c. If VERIFY then Establish Video/audio link if location is “wired”
   d. If VERIFY then Alarm Staff/Security PDAs
   e. Document Event
   f. If 30 seconds elapse with no response from Nursing Station then d. & e.
2. If [X]=1 and solo Resident RFID/ultrasound (no associated Staff or Family RFID/ultrasound) is detected outside room [F] then
   a. Alert Nursing Station
   b. Wait 30 seconds for VERIFY/REJECT
   c. If VERIFY then Establish Video/audio link if location is “wired”
   d. If VERIFY then Alarm Staff PDAs
   e. Document Event
   f. If 30 seconds elapse with no response from Nursing Station then d. & e.
3. If Resident RFID/ultrasound is detected in any Facility Area that is denoted “Restricted” without the presence of Staff RFID/ultrasound then
   a. Alert Nursing Station
   b. Wait 30 seconds for VERIFY/REJECT
   c. If VERIFY then Establish Video/audio link if location is “wired”
   d. If VERIFY then Alarm Staff/Security PDAs
   e. Document Event
   f. If 30 seconds elapse with no response from Nursing Station then d. & e.

“Closest Staff Locator”/Shell Method

For a uniform grid of RFID/ultrasound zones measuring MM by NN and numbered from left to right, starting in the upper left zone
Shell 0=X (the present location of the Resident in need)
Shell 1=X−1, X+1, X+MM, X−MM, X+MM+1, X+MM−1, X−MM+1, X−MM−1
Shell 2=X−2, X+2, X−2+MM, X−2−MM, X−2+2MM, X−2−2MM, X+2+MM, X+2−MM, X+2+2MM, X+2−2MM, X−2MM, X−2MM+1, X−2MM−1, X+2MM, X+2MM+1, X+2MM−1.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. In a facility that includes a plurality of patients, a support upon which each patient rests at least some of the time, and a computer-controlled system for monitoring positions and/or movements of the patients that are predictive of patient support exiting and providing an appropriate response thereto, a method for monitoring a patient while resting on a support and determining whether the patient is likely preparing to exit the support, comprising:
accessing by a computer system a patient profile associated with the patient being monitored from among a plurality of different patient profiles corresponding to different patients, the patient profile including data relating to at least one position and/or movement of the patient that is predictive of support exiting by the patient and that differs from profile data relating to support exiting by at least one other patient;
the computer system generating and/or receiving a data stream representative of one or more positions and/or movements of the patient;
the computer system comparing the data stream representative of one or more positions and/or movements of the patient and comparing it with patient profile data that has been predetermined to be predictive of support exiting by the patient being monitored; and
initiating a response in order to prevent or mitigate harm to the patient upon finding a correlation between the data stream representative of one or more positions and/or movements of the patient and the patient profile data that has been predetermined to be predictive of support exiting by the patient being monitored.

2. The method as recited in claim 1, wherein the computer system generating and/or receiving a data stream representative of one or more positions and/or movements of the patient comprises the computer systems system generating and/or receiving a data stream in response to an ultrasound detector detecting an ultrasound signal from an ultrasound device attached to the patient.

3. In a computer-controlled system for monitoring one or more positions and/or movements of a patient on a support that are predictive of patient support exiting and providing an appropriate response thereto, a method for monitoring the patient while resting on the support and determining whether the patient is likely preparing to exit the support, comprising:
a computer system accessing a patient profile associated with the patient being monitored that includes data relating to at least one position and/or movement of the patient that has been predetermined to be predictive of support exiting by the patient;
the computer system receiving a video data stream of the patient while resting on the support;
the computer system analyzing the video data stream, determining at least one position and/or movement of the patient from the video data stream, and comparing the at least one position and/or movement of the patient determined from the video data stream with patient profile data that has been predetermined to be predictive of support exiting by the patient; and
the computer system initiating a response in order to prevent or mitigate harm to the patient upon finding a correlation between the at least one position and/or movement of the patient determined from the video data stream and the patient profile data.

4. A method as defined in claim 3, the patient profile data relating to predicted support exiting by the patient being monitored differing from patient profile data relating to predicted support exiting by at least one other patient.

5. A method as defined in claim 1 or 4, the patient profile data of the patient being monitored differing from profile data for at least one other patient with respect to one or more of the following movements relating to potential bed exiting behavior:
(a) movement towards the bottom of a bed;
(b) right side bedrail roll;
(c) left side bedrail roll;
(d) torso angle up and leg swing right;
(e) torso angle up and leg swing left;
(f) torso angle up and upper body roll right; and/or
(g) torso angle up and upper body roll left.

6. A method as defined in claim 1, the data stream comprising a video data stream generated by at least one camera fixed on the patient while resting on the support.

7. A method as defined in claim 6, the video data stream being generated by a plurality of cameras, at least one of which is positioned so as to provide an aerial view of the patient and support and at least one other of which is positioned so as to provide a side view of the patient and support.

8. A method as defined in claim 6, at least one camera being positioned so as to provide a view of a door or other entrance to where the patient is located.

9. A method as defined in claim 6, at least one camera being positioned near a video monitor that is viewable by the patient, wherein the response includes transmitting an image of a person to the video monitor.

10. A method as defined in claim 1, the data stream being generated by a light beam matrix system comprising a plurality of spaced apart light emitters and corresponding light sensors that are positioned above and beside the patient being monitored.

11. A method as defined in claim 1, the data stream being generated by a small zone RFID grid system comprising a plurality of spaced apart RFID detectors adjacent to the patient being monitored and a plurality of RFID devices worn by the patient.

12. A method as defined in claim 1, the data stream being generated by a small zone ultrasound grid system comprising a plurality of spaced apart ultrasound detectors adjacent to the patient being monitored and a plurality of ultrasound devices worn by the patient.

13. A method as defined in claim 3 or 6, the video data stream being buffered by the computer system and then deleted from the computer system upon being analyzed in the absence of an archival triggering event in order to preserve patient privacy.

14. A method as defined in claim 13, upon the occurrence of an archival triggering event, the video data stream being archived for later viewing.

15. A method as defined in claim 1, the response including transmitting at least a portion of a video stream of the patient to a video monitor at a central station for confirmation or denial by staff of behavior consistent with patient support exiting.

16. A method as defined in claim 15, the confirmation or denial of behavior consistent with patient support exiting being used to update the patient profile in order for the computer system to more accurately determine future support exiting by the patient.

17. A method as defined in claim 15, upon confirming behavior consistent with patient support exiting by a staff member, the response further including establishing two-way audio-video communication between the staff member and the patient.

18. A method as defined in claim 15, upon confirming behavior consistent with patient support exiting, the response further including sending an alert to a caregiver able to provide assistance to the patient.

19. A method as defined in claim 15, in the absence of confirmation or denial of behavior consistent with support exiting within a predetermined time period, the response further including transmitting a pre-recorded message to the patient.

20. A method as defined in claim 15, upon confirming behavior consistent with patient support exiting, the response further including at least one of bed lowering, raising a bedrail, turning on a light, or actuation of a patient restraint device in order to prevent or mitigate potential harm to the patient.

21. A method as defined in claim 15, further comprising providing an alert to the patient that the video data stream is available for viewing by a third party.

22. A method as defined in claim 1 or 3, further comprising the computer receiving and analyzing biometric data relating to the patient.

23. A method as defined in claim 1, the support being selected from a bed, bed with side rails, wheelchair, gurney, couch, chair, or recliner.

24. A system for monitoring one or more positions and/or movements of a patient on a support that are predictive of preparation for patient support exiting and providing an appropriate response thereto comprising means for performing the method of claim 1.

25. A computer-program product comprising one or more computer-readable media having stored thereon computer-executable instructions that, when executed by a processor of a computer system, causes the computer system to perform the method of claim 3.

26. In a facility that includes a plurality of patients, supports upon which the patients rest at least some of the time, and a computer-controlled system for monitoring one or more positions and/or movements of each patient that are predictive of patient support exiting and providing an appropriate response thereto, a method for monitoring the plurality of patients while resting on the supports and determining whether one or more of the patients are likely preparing to exit a support, comprising:
inputting into a computer system a plurality of patient profiles that include data relating to at least one position and/or movement of each patient that has been predetermined to be predictive of support exiting by that patient, at least two of the patient profiles differing with respect to data relating to predicted support exiting in order to more accurately distinguish, as between different patients, behavior that is predictive of support exiting from non support exiting behavior;
sending video data streams of two or more patients while resting on respective supports to the computer system;
the computer system analyzing the video data streams, determining at least one position and/or movement of each patient from the video data streams, and comparing the at least one position and/or movement of each patient determined from the video data streams with corresponding patient profile data that has been predetermined to be predictive of support exiting by that patient; and
initiating a response in order to prevent or mitigate harm to a patient upon finding a correlation between the at least one position and/or movement of the patient determined from the video data stream and patient profile data corresponding to the patient.

27. A method as defined in claim 26, the computer system including a facility master computer and a plurality of in room controllers, the facility master computer storing and periodically updating the plurality of patient profiles, the in room controllers being associated with respective rooms containing respective patients being monitored, each in room controller accessing profile data and analyzing a video data stream corresponding to a patient being monitored in a respective room.

28. A method as defined in claim 27, the computer system including ultrasound sensors coupled to the in room controllers to monitor patients.

29. In a facility that includes a patient, a bed upon which the patient rests at least some of the time, and a computer-controlled system for monitoring one or more positions and/or movements of the patient that are predictive of patient bed exiting and providing an appropriate response thereto, a method for monitoring the patient while resting on the bed and determining whether the patient is likely preparing to exit the bed, comprising:
inputting into a computer system a patient profile associated with the patient being monitored that includes data relating to one or more of the following movements that are predictive of potential bed exiting by the patient:
(a) movement towards the bottom of a bed;
(b) righte side bedrail roll;
(c) left side bedrail roll;
(d) torso angle up and leg swing right;
(e) torso angle up and leg swing left;
(f) torso angle up and upper body roll right; and/or
(g) torso angle up and upper body roll left;
sending a video data stream of the patient while resting on the bed to a computer system;
the computer system analyzing the video data stream, determining at least one movement of the patient from the video data stream, and comparing the at least one movement of the patient determined from the video data stream with one or more movements in the patient profile; and
initiating a response in order to prevent or mitigate harm to the patient upon finding a correlation between the at least one movement of the patient determined from the video data and the one or more movements in the patient profile.

30. The method as recited in claim 29, wherein inputting into a computer system a patient profile associated with the patient being monitored comprises inputting a patient profile associated with a patient that is being monitored by ultrasound sensors.

31. A patient monitoring system comprising means for performing the method of claim 26 or 29.
